(19) United States
(12) Patent Application Publication
(10) Pub. No.: US 2013/0156785 A1
(43) Pub. Date: Jun. 20, 2013

(54) NOVEL DIAGNOSTIC AND THERAPEUTIC TARGET IN INFLAMMATORY AND/OR CARDIOVASCULAR DISEASES

(71) Applicants: Chad Brokopp, Zurich (CH); Simon Hoerstrup, Zurich (CH)

(72) Inventors: Chad Brokopp, Zurich (CH); Simon Hoerstrup, Zurich (CH)

(73) Assignee: UNIVERSITY OF ZURICH, Zurich (CH)

(21) Appl. No.: 13/819,335

(22) PCT Filed: Aug. 29, 2011
(86) PCT No.: PCT/EP2011/064807

§ 371 Date: Feb. 26, 2013

Related U.S. Application Data
(60) Provisional application No. 61/377,663, filed on Aug. 27, 2010.

(30) Foreign Application Priority Data
Aug. 27, 2010 (EP) 10008937.4

(51) Int. Cl. G01N 33/68; C07K 16/40; C12Q 1/56; A61K 31/713; A61K 38/48; C12Q 1/68; A61K 38/55

(52) U.S. Cl. CPC G01N 33/6893; C12Q 1/6876; C07K 16/40; A61K 38/55; A61K 31/713; A61K 38/482; C12Q 1/56
USPC 424/142.1; 435/7.21; 435/6.11; 435/6.12; 424/146.1; 514/20.3; 514/44 A; 424/94.64; 435/13

(57) ABSTRACT

Methods for diagnosing inflammatory and/or cardiovascular diseases by assaying for Fibroblast Activation Protein (FAP) expression in a body fluid is provided as well as therapeutic means based thereon.

### FIELD OF INVENTION

The present invention relates to methods and compositions for identifying, diagnosing, evaluating or treating an inflammatory disease or condition in a subject. In addition, the present invention relates to a novel therapeutic and diagnostic target in cardiovascular diseases.

### BACKGROUND OF THE INVENTION

Cardiovascular disease and inflammatory disease is the leading cause of death in an industrialized country and is emerging as the major cause of death in developing countries. While current clinical techniques such as, but not limited to, diagnostic coronary angiograms, doplar, echocardiography, and blood-based biomarker analysis are employed to diagnose vascular disease, these technique often cannot discriminate between stable and clinically significant cardiovascular disease which may justify therapeutic interventions. Indeed cardiovascular diseases most frequently go undiagnosed before clinical intervention or death. Moreover, many of these diagnostic and therapeutic techniques are inaccessible to much of the global population, due to unaffordable costs and technically demanding intervention procedures. Therefore, an unmet clinical need remains for more precise, cost-effective, and technically simplified diagnostic and therapeutic techniques to improve outcomes and overall costs associated with cardiovascular disease.

Chronic inflammatory diseases, such as coronary artery disease starts with the formation of atherosclerotic plaques in the coronary arteries. Abrupt occlusion of these atherosclerotic arteries due mainly to thrombosis leads to coronary heart diseases: unstable angina, acute myocardial infarction and sudden death. Coronary artery disease is a disease of several risk factors, most notably among which are hyperlipidemia, hypertension, diabetes mellitus and tobacco smoking. Multiple pathogenic factors are known to be operating at the cellular level, such as enhanced oxidation of low-density lipoprotein (LDL) and proliferation of monocyte-derived macrophages and smooth muscle cells (SMCs). Atherogenesis is a complex process involving, among others, endothelial cell dysfunction such as increased endothelial permeability to lipoproteins. The origin or cause of all stages of atherosclerotic cardiovascular diseases has been implicated by inflammation and is considered to be a major part of the pathophysiological basis of atherogenesis. Atherosclerosis is a degenerative inflammatory process that affects artery walls. Due to the lack of appropriate diagnostic markers, the first clinical presentation of more than half or the patients with coronary artery diseases is either myocardial infarction or death.

Rupture of the fibrous cap in so-called “vulnerable atherosclerotic plaques” is a critical trigger of myocardial infarction and stroke. Thereby vulnerable atherosclerotic plaques become thrombogenic by either rupturing open, or expressing pro-thrombotic agents which promote blood coagulation and occlude the coronary blood flow. Atherothrombosis is a term which describes the blood coagulation derived from atherosclerotic plaques to form so-called occluding “coronary thrombi”. One of the key events involved in promoting plaque instability is degradation of the fibrous cap, which exposes the underlying thrombogenic plaque core to the bloodstream, thereby causing thrombosis and subsequent vessel occlusiont³ Vulnerable plaque rupture is facilitated by proteases which cleave collagen, the primary load-bearing molecule in fibrous caps.⁴⁻⁷

Coronary thrombus formation is orchestrated by myriad proteases whose enzymatic activities promote thrombosis via blood clotting. An understanding of the specific proteins which are expressed on each cell population may allow for identification of novel biomarkers and therapeutic targets against myocardial infarction. Moreover, as myocardial infarctions often reoccur, a complete protein/cell description of individuals' thrombi may motivate personalized preventative treatment against reoccurring events. Unfortunately, the proteins expressed by specific cell populations in coronary thrombi remain unknown.

Therefore, activated proteases which degrade the fibrous cap have gained attention as potential diagnostic and therapeutic targets. Candidate targets include matrix metalloproteinases (MMP)-2 and 9 and the cysteine protease cathepsin K; each of which are enhanced in both stable and unstable lesions.⁸⁻¹¹ MMP-2 and cathepsin K staining reveal diffuse localization throughout the plaque, whereas MMP-9 colocalizes with macrophages beneath the fibrous cap.¹²⁻¹⁴

While MMPs and cysteine proteases have shown potential as markers of atherosclerotic plaques, their diffuse expression in all lesions warrants careful assessment of their targeting potential toward clinically relevant unstable plaques. An ideal target would be specific to the rupture-prone fibrous cap; a site perhaps more easily accessible by an intravenously injected targeting agent.

Thus, there is a need for novel therapeutic and diagnostic markers and non-invasive methods in order to diagnose and treat an inflammatory and/or a cardiovascular disease in a subject. The solution to said technical problem is achieved by providing the embodiments as characterized in the claims and described further below.

### SUMMARY OF THE INVENTION

Object of the present invention are a method for determining the presence, onset or progression of inflammatory and/or cardiovascular diseases and conditions and identifying novel targets for diagnosing or treating inflammatory and/or cardiovascular diseases such as rheumatoid arthritis, acute coronary syndrome and atherothrombosis.

The present invention is based on a novel and surprising finding that Fibroblast Activation Protein (FAP) is induced by macrophage derived Tumour necrosis alpha (TNF-α) in human aortic smooth muscle cells (HASMC) and associated with vulnerable human plaques and contributes to collagen breakdown in rupture prone fibrous caps. The present invention also provides evidence that Fibroblast Activation Protein (FAP) plays a role in arthritis and tumor formation through its collagenase activity. Furthermore, the present invention demonstrates that FAP expression or activity can be neutralized by blocking agents such as anti-FAP antibodies.

In particular, the present invention makes use of the surprising finding that FAP protein is expressed or increased expressed in a sample of a patient suffering from an inflammatory disease and/or cardiovascular disease compared to the FAP expression in a healthy control sample. Put in other words, a method of determining the presence of an inflammatory disease and/or cardiovascular disease or condition in a patient is provided comprising assaying a sample taken from said patient for expression of FAP, wherein expression or an increased expression of said FAP compared to a control sample is indicative for the disease or condition in said patient. Preferably, said sample is a body fluid, most preferably blood.

More specifically, the present invention relates to a method for determining the presence of vulnerable atherosclerotic plaques or atherothrombosis, wherein a sample, preferably a sample of or derived from blood, thrombus or plaque is diagnosed with the help of immuno- and nucleic acid based assays such as the use of antibodies and/or RT-PCR.

The invention also relates to a composition for the diagnosis and/or treatment of an inflammatory and/or a cardiovascular disease in a mammal which has been determined for FAP expression. In particular, the present invention relates to a composition comprising a compound capable of (i) detecting the presence or activity of FAP and/or (ii) inhibiting the activity of FAP or its expression for use in the diagnosis or treatment of a disease or condition. The composition of the present invention may be (i) a pharmaceutical composition and comprises a pharmaceutically acceptable carrier or (ii) a diagnostic composition and optionally comprises suitable means for detection of the compound and/or of FAP.

More specifically, the composition is capable of inhibiting, reducing or lowering the enzymatic activity of FAP protein, in particular capable of inhibiting coagulation, i.e. blood clotting. Furthermore, the compound of the composition according to the present invention may be able to associate with FAP either with the protein or fragment thereof or with the nucleic acid molecule coding for FAP or fragments thereof. Therefore, the compound may be or not be a small molecule, an antibody or an antagonist, preferably a peptide or a peptide analog. In a further aspect, the present invention makes use of the above described method to identify and diagnose a patient to be treated by the composition according to the present invention.

In a further aspect, the present invention relates to a composition comprising FAP for the use in the prevention or treatment of a coagulation disorder, in particular hemophilia. The present invention also provides the use of FAP as a reagent for enhancing coagulation, in particular blood clotting ex vivo.

### BRIEF DESCRIPTION OF THE DRAWINGS

FIG. 1: FAP expression is enhanced in human atherosclerotic aortic plaques. A, Movat and FAP stainings shows representative cross sections of plaque-free aortae and fibroatheromata (L=lumen, M=media, P=atherosclerotic plaque; bar=400 μm). Dotted boxes indicate regions of interest in adjacent sections at high magnification (bar=50 μm). B, Immunofluorescence stainings in representative tissue sections of plaque-free aortae, type II-III plaques, and type V plaques show FAP expression in red (DAPI in blue; bar=50 μm). C, The graph reveals a significant increase in FAP expression in Type II-III aortic plaques (n=9) and in Type IV-V plaques (n=12) compared with plaque-free aortae (n=8).

FIG. 2: FAP expression in human aortic plaques colocalizes with smooth muscle cells, but not with macrophages or endothelial cells. A, Overlays of confocal images of FAP (red) and DAPI (blue) with cell-specific stainings of αSMA, CD68, and vWF (green) in representative sections illustrate FAP colocalization (arrows) with smooth muscle cells (bar=20 μm). B, The graph quantifies an increased colocalization of FAP with smooth muscle cells (αSMA), compared with endothelial cells (vWF) and macrophages (CD68) in type V atherosclerotic plaques (n=10).

FIG. 3: FAP is constitutively expressed in cultured human aortic smooth muscle cells (HASMC) and endothelial cells (HAEC), but not in peripheral blood-derived monocytes (PBM), macrophages (MΦ), or foam cells. FACS analyses and Oil-Red-O staining characterize cells populations (top panel) and their respective FAP expression (bottom panel).

FIG. 4: FAP expression is enhanced in “unstable” thin-cap versus “stable” thick-cap human coronary fibroatheromata. A, Masson staining shows collagen-rich “stable” thick (658 μm) versus “unstable” thin (45 μm) fibrous caps (L=lumen, FC=fibrous cap, NC=necrotic core; bar=1 mm). FAP immunohistochemistry and immunofluorescence (intensity scale; bar=50 μm) shows FAP expression in representative thin vs. thick caps. Dotted boxes indicate regions of interest in adjacent sections at high magnification. B, The graph reveals a significant increase in FAP expression in thin vs. thick fibrous caps (n=12 each).

FIG. 5: FAP expression correlates with macrophage burden in human aortic plaques. A, Confocal immunofluorescent photomicrographs of a representative aortic fatty streak reveals FAP expression (red) adjacent to macrophages (CD68; green) at low (phase contrast in white; bar=100 μm) and high (bar=25 μm) magnification. B, Greyscale photomicrographs of FAP or macrophage (CD68) immunofluorescent stainings in plaque-free aortae, Type II, and Type V atherosclerotic plaques show enhanced FAP expression with increasing macrophage burden (bar=50 μm). C, Comparisons of FAP and macrophage (CD68) expression in serial adjacent sections from aortic plaques demonstrate a significant positive correlation (R²=0.763; n=12; p<0.05); AU indicates arbitrary units.

FIG. 6: Macrophage-derived TNFα induces FAP expression in HASMC. A, Macrophage-conditioned supernatant induces FAP in HASMC in a concentration-dependent manner following 48 hr exposure (n=6). B, Using the same macrophage-conditioned medium, TNFα-blocking antibody (Ab6671) decreases FAP expression by 40% in HASMC compared with an isotype control antibody (n=6). C, Recombinant human TNFα induces FAP in HASMC in a dose-dependent manner after 48 hr incubation (n=6). D, Recombinant human TNFα induces FAP in HASMC in a time-dependent manner (30 ng/mL). AU indicates arbitrary units (*=p<0.05, **=p<0.01, NS=not significant).

FIG. 7: Gelatinase activity is inhibited in human aortic fibrous caps by the FAP blocking antibody A246. A, Movat staining of a representative fibrous cap in human aortic fibrous cap. The region of interest (black box) is shown at higher magnification in an adjacent section stained for FAP (red), type I collagen (green), and overlay (DAPI=blue; bar=150 μm). B, The graph reveals dose dependant inhibition of rhuFAP gelatinase activity by A246 (n=6/group) C, Confocal images of in situ zymography show FAP (red) and cleaved type I collagen (green) in fibrous caps of aortic plaque treated with a control IgG antibody or A246 (bar=10 μm). D, The graph reveals a significant reduction of cleaved collagen colocalized with FAP expression (n=10/group).

FIG. 8: A Sandwich ELISA has been established to quantify soluble FAP in human peripheral blood. The standard curve reveals a linear relationship between FAP protein concentration and fluorescent signal.

FIG. 9: FAP accelerates the rate of human blood clotting. Representative ROTEM readouts from a NATEM assay treated with increasing concentrations of recombinant human FAP over increasing incubation periods are shown. Peripheral blood is harvested in citrate tubes from healthy volunteers and conditioned with recombinant human FAP for the indicated dosages and times.

FIG. 10: FAP accelerates clotting time and clot formation time and enhances alpha angle and maximum clot firmness. Blood from 6 healthy volunteers with plasma FAP levels below 20 ng/mL has been harvest and conditioned with recombinant human FAP at the doses and incubation times indicated. Average values and standard deviations compared to unconditioned controls are shown (*, p<0.05; **, p<0.01, Student's T-test). Changes in maximum clot firmness is not significant.

FIG. 11: FAP accelerates clotting time and clot formation time and enhances both alpha angle and maximum clot firmness by NATEM. Absolute clotting time and clot formation time decreased in 5/6 probands, alpha angle increased in 5/6 patients, and maximum clot thickness increased in 4/6 probands when conditioned with 1.75 ug/mL of FAP at 5 hr incubation compared to vehicle treated control.

FIG. 12: FAP expression is enhanced in aortic atherosclerotic plaques of ApoE^{-/-} mice. A, H&E staining of aortic root cross sections from wildtype and atherosclerotic ApoE^{-/-} mice (bar=300 μm) are shown at higher magnifications (inlays) for regions of interest (black box; bar=50 μm). Adjacent immunofluorescence stainings reveal enhanced FAP expression (red) and macrophage burden (CD68; green) in the plaque-bearing root compared with wild type controls (DAPI in blue). B, Quantitative image analysis reveals increased FAP signal intensity in plaques (n=5) compared with wild-type (n=4) controls (*p<0.002; Mann-Whitney U-test). AU indicates arbitrary units.

FIG. 13: FAP is expressed in the endothelium of human thin-cap coronary fibroatheromata. H&E staining shows an internal mammary artery and a thin-cap coronary fibroatheromata (L=lumen, FC=fibrous cap, NC=necrotric core; bar=1 mm). FAP and CD31 immunohistochemistry (bar=50 μm) shows FAP expression in the endothelium (arrows). Dotted boxes indicate regions of interest in adjacent sections at high magnification, and stainings with isotype control antibodies are shown.

FIG. 14: FAP expression is enhanced in human coronary thrombi compared to peripheral blood. FAP is enhanced in human coronary thrombi vs. peripheral blood in STEMI patients. Representative immunohistological stainings illustrate enhanced FAP in coronary thrombi vs. peripheral blood.

FIG. 15: FAP is enhanced in human coronary thrombi granulocytes compared to peripheral blood. Representative gating by flow cytometry of monocytes (A), T-lymphocytes (B), and granulocytes (C) are shown. Analysis of peripheral blood (PB) vs coronary thrombus (TH) cell populations reveals low FAP expression levels in monocytes (D) and T-lymphocytes (E), and a significant increase of FAP expression in granulocytes (F; n=11, Paired Student's T-Test).

FIG. 16: Granulocyte FAP expression is enhanced in acute, but not chronic thrombosis. Granulocyte FAP expression is enhanced in peripheral blood (white bar) and occluding thrombi (grey bar) from patients with myocardial infarction (MI, n=11) compared to patients with peripheral artery occlusive disease (PAOD; n=4; mean; +/− standard deviation; Student's T-Test). Plasma FAP levels are enhanced in patients with acute coronary syndromes. FAP levels were significantly enhanced in patients with Stable CAD compared to patients with No CAD. FAP levels were further enhanced in patients with unstable angina and with STEMI compared to patients with no CAD or stable CAD (values represent mean+/− Standards deviation; Mann Whitney U-Test).

FIG. 17: Plasma FAP levels are enhanced in patients with acute coronary syndromes. FAP levels were significantly enhanced in patients with Stable CAD compared to patients with No CAD. FAP levels were further enhanced in patients with Unstable angina and with STEMI compared to patients with no CAD or stable CAD (values represent mean+/− Standards deviation; Unpaired Student's T-Test).

### DEFINITIONS AND GENERAL TECHNIQUES

Unless otherwise stated, a term as uses herein is given the definition as provided in the Oxford dictionary of biochemistry and molecular biology, Oxford University Press, 1997, revised 2000 and reprinted 2003, ISBN 0 19 850673 2.

For further elaboration of general techniques useful in the practice of this invention, the practitioner can refer to standard textbooks and reviews in cell biology and tissue culture; see also the references cited in the examples. General methods in molecular and cellular biochemistry can be found in such standard textbooks as Molecular Cloning: A Laboratory Manual, 3rd Ed. (Sambrook et al., Harbor Laboratory Press 2001); Short Protocols in Molecular Biology, 4th Ed. (Ausubel et al. eds., John Wiley & Sons 1999); Protein Methods (Bollag et al., John Wiley & Sons 1996); Non-viral Vectors for Gene Therapy (Wagner et al. eds., Academic Press 1999); Viral Vectors (Kaplitt & Loewy eds., Academic Press 1995); Immunology Methods Manual (Lefkovits ed., Academic Press 1997); and Cell and Tissue Culture: Laboratory Procedures in Biotechnology (Doyle & Griffiths, John Wiley & Sons 1998). Reagents, cloning vectors and kits for genetic manipulation referred to in this disclosure are available from commercial vendors such as BioRad, Stratagene, Invitrogen, Sigma-Aldrich, and ClonTech.

“Agent”, “reagent” or “compound”, are the terms as used herein, generally refer to any substance, chemical composition or extract that have a positive or negative biological effect on a cell, tissue, body fluid or within the context of any biological system, or any assay system examined. They can be antagonists, agonists, partial agonists or inverse agonists of a target. Such agents, reagents or compounds might be nucleic acid, natural or syntactic peptides or protein complexes, or fusion proteins. They may also be antibodies, organic or inorganic molecules or compositions, small molecules, drugs and any combinations of any of said agents above. They may be used for testing, for diagnostic or for therapeutic purpose.

If not stated otherwise the terms “composition” are used interchangeably herein and include but are not limited to therapeutic agents (or potential therapeutic agents), food additives and nutraceuticals. They can also be animal therapeutics or potential animal therapeutics.

“Treatment”, “treating” and the like are used herein to generally mean obtaining a desired pharmacological and/or physiological effect. The effect may be prophylactic in terms of completely or partially preventing a disease or symptom thereof and/or may be therapeutic in terms of partially or completely curing a disease and/or adverse effect attributed to the disease.

“Inhibitor” is any substance which retards or prevents a chemical or physiological reaction or response.

The terms “induce”, “inhibit”, “increase”, “decrease” “lower” or the like, e.g., which denote quantitative differences between two states, refer to at least statistically significant differences between the two states. For example, “a compound capable of inhibiting the activity or expression of FAP” means that the level of activity or expression of FAP in a treated sample will differ statistically significantly from the level of FAP activity or expression in untreated cells. As used herein, “inhibiting the expression or activity” of FAP refers to a reduction or blockade of the expression or activity, e.g., enzymatic activity and does not necessarily indicate a total elimination of the FAP expression or activity. Such terms are applied herein to, for example, levels of expression, and levels activity.

“Standard expression” is a quantitative or qualitative measurement for comparison. It is based on a statistical appropriate number of normal samples and is created to use a basis of comparison when performing diagnostic assays, running clinical trials or falling patient treatment profiles.

“Subject” as used herein might be defined to include human, domestic (e.g. cats, dogs etc.), agriculture (e.g. cows, horses, sheep etc.) or test species (e.g. mouse, rat, rabbit etc.).

The term “sample” as used herein refers to a biological sample obtained for the purpose of evaluation in ex vivo or in vitro. The patient and control sample may be discarded afterwards or stored under appropriate conditions until future use. Thereby the stored sample may be used for further analysis or comparison means. The patient sample is solely used for the in vitro diagnostic method of the invention and the material of the patient sample is not transferred back into the patient's body.

Herein the term “body fluid” relates to fluids e.g. blood, sputum, urine, ascetic fluid, pleural fluid, spinal fluid, lymph, serum, mucus, saliva, semen, ocular fluid, extracts of nasal, throat or genital swabs wherein cells or proteins and their respective fragments, catalytic products and precursors are dissolved in a fluid and circulate within the body. In one embodiment fluids and cells which do not circulate, i.e. being rather stationary located in the body such as myofibroblast-like synoviocytes are preferably excluded from the method of the present invention.

“Inflammatory disease” originates out of an inflammatory process. Inflammation is part of the innate immune response that occurs in reaction to any type of bodily injury. Inflammation has very specific characteristics, whether acute or chronic, and the innate immune system plays a pivotal role, as it mediates the first response. Infiltration of innate immune system cells, specifically neutrophils and macrophages, characterizes the acute inflammation, while infiltration of T lymphocytes and plasma cells are features of chronic inflammation. Monocytes/macrophages play a central role in both, contributing to the final consequence of chronic inflammation which is represented by the loss of tissue function due to fibrosis. In some disorders, the inflammatory process—which under normal conditions is self-limiting—becomes continuous and chronic inflammatory diseases might develop subsequently. Inflammatory disease is herein preferably understood as a chronic inflammatory diseases such as inflammatory bowel disease, coronary artery disease, forms of arthritis, including rheumatoid arthritis, ankylosing spondylitis and osteoarthritis; tendinitis or tenosynovitis; inflammatory myopathies; inflammatory neuropathies; multiple sclerosis; epilepsy; inflammatory site edema; post-event ischemia and reperfusion symptomlogy resulting from acute central nervous system trauma, including stroke and spinal cord trauma; post-event consequences of kidney ischemia and reperfusion; acne vulgaris; asthma; chronic prosatitis; glomerulonephritis; pelvic inflammatory disease; transplant rejection; vasculitits; and post-event consequences of reperfusion subsequent to myocardial infarction. In this context, the term “inflammatory disease” also includes conditions which are associated with inflammatory conditions such as referred to above, for example atherosclerosis and atherosclerotic plaque, and which otherwise may be regarded as cardiovascular diseases. Thus, in accordance with the present invention inflammatory disease is used herein to relate to chronic inflammatory diseases including cardiovascular diseases and rheumatoid arthritis. For further reading see Stitzinger “Lipids, inflammation and atherosclerosis” (pdf). The digital repository of Leiden University. https://openaccess.leidenuniv.nl/dspace/bitstream/1887/9729/11/01.pdf. (2007).

The term “cardiovascular diseases” or “disorder” includes heart disorders, as well as disorders of the blood vessels of the circulation system caused by, e.g., abnormally high concentrations of lipids in the blood vessels.

As used herein, the term “atherosclerosis” is intended to have its clinical meaning. This term refers to a cardiovascular condition occurring as a result of lesion (e.g., plaque or streak) formation in the arterial walls. The formation of plaques or streaks results in a reduction in the size of the inner lining of the arteries. These plaques consist of foam cells filled with modified low-density lipoproteins, oxidized-LDL, decaying smooth muscle cells, fibrous tissue, blood platelets, cholesterol, and sometimes calcium. They tend to form in regions of disturbed blood flow and are found most often in people with high concentrations of cholesterol in the bloodstream. The number and severity of plaques increase with age thereby promoting thrombus formation (blood clots).

As used herein “atherosclerotic plaque” consists of accumulated intracellular and extracellular lipids, smooth muscle cells, connective tissue, and glycosaminoglycans. The earliest detectable lesion of atherosclerosis is the fatty-streak lesion comprising a lipid-laden foam cells, which are macrophages that have migrated as monocytes from the circulation into the subendothelial layer of the intima, which later involves into the fibrous plaque, consisting of intimal smooth muscle cells surrounded by cognitive tissue and intracellular and extracellular lipids. As plaques develop, calcium is deposited. Acute coronary events manifest when atherosclerotic plaque ruptures and blood comes into contact with the plaque's prothrombogenic core.

“Vulnerable atherosclerotic plaque” is an atherosclerotic plaque which is prone thrombotic processes. A characteristic of vulnerable plaque prone to rupture is a lipid core covered by a thin fibrous cap and inflammatory cells. Plaques with a thin fibrous cap, often than 50-μm thick, lose their stability and become unable to withstand haemodynamic stress in circumferential, radial, and/or shear direction, which leads subsequent rupture or leakage of the prothrombotic material into the vessel lumen. The amount of lipid and composition of the lipid pool also promote plaque instability. Inflammation is a third factor affecting plaque vulnerability. Macrophages infiltrate the vessel wall and release proteases capable of degrading the extracellular matrix. Thinning of the fibrous cap is therefore at the basis of atherosclerotic plaque rupture.

### DETAILED DESCRIPTION OF THE INVENTION

The present invention generally relates to means and methods and compositions for research, prevention and treatment of inflammatory and/or cardiovascular diseases or conditions like vulnerable atherosclerotic plaques and/or atherothrombosis, wherein the Fibroblast Activation Protein (FAP) expression or activity is determined and indicative for the disease or condition in a patient. As demonstrated in the Examples, it was surprisingly found in accordance with the present invention that determination of expression and/or increased expression or activity of FAP enzyme, e.g. the enzymatic activity of a FAP protein and/or fragment or catalytic product of said protein in a body fluid sample allows the assessment of an inflammatory and/or cardiovascular disease. In addition, it was found that an increased expression and/or activity of FAP protein or fragments thereof in a sample compared to a control such as obtained from a healthy volunteer is indicative for the risk or progression of said diseases.

Fibroblast activation protein (FAP) is a membrane-bound, constitutively active serine protease expressed by activated fibroblasts in epithelial tumor stroma, arthritis, and wound healing, but remains virtually undetectable in healthy tissues.¹⁵⁻¹⁷ FAP is an enzyme that exhibits dipeptidyl peptidase IV activity, prolyl endopeptidease activity, and type I collagen specific activity.¹⁷⁻¹⁹ The nucleotide and amino acid sequence of human FAP are known in the art and can be obtained via public databases, for example, the internet pages hosted by the National Centre for Biotechnology Information (NCBI), including the NIH genetic sequence database Genebank, which also cites the corresponding references available by PubMed. Central. For example, the human nucleotide and amino acid sequence of Fibroblast Activation Protein (FAP) are available under accession number NM_{—}004460 and AAB_{—}496652.1. In accordance with the experimental data described infra, one object of the present invention is to characterize FAP expression in human atherosclerosis and examine its association with plaque instability in order to provide methods and compositions to diagnose and treat a subject suffering from an FAP associated inflammatory disease and/or cardiovascular disease.

Collagen degradation in vulnerable atherosclerotic plaques renders them more prone to rupture. Fibroblast Activation Protein (FAP) plays a role in tumor formation through its collagenase activity. However, the significance of FAP in vulnerable human plaques hitherto remained unknown.

Therefore, one object of the present invention is to characterize FAP expression in human atherosclerosis and examine its association with plaque instability in order to provide a method capable of diagnosing and treating a subject suffering from said disease. Moreover, another object of the present invention is to determine FAP's mechanism of induction, its downstream effects, and the neutralizing capacity of antibodies against FAP expression and activity.

In particular, it could surprisingly be shown that FAP is expressed in rupture prone coronary plaques and endothelial cells in patients with acute coronary syndrome (ACS). The present invention is based on the observations that FAP mediated collagenolysis is induced by inflammatory signaling and can be neutralized by inhibitors or antagonists of FAP protein. The present invention also provides the surprising finding that FAP is induced by macrophage-derived TNFα in HASMC, associates with vulnerable human plaques, and contributes to collagen breakdown in rupture-prone fibrous caps.

Thanks to the present invention, for the first time a method can be provided in order to characterize an inflammatory and/or cardiovascular disease, respectively, at an earlier stage i.e. while the disease is still clinically silent. Therefore, the present invention provides FAP as a biomarker for diagnosing the grade of progression and vulnerability of atherosclerotic plaque in a subject suffering from an atherosclerosis-associated disease.

Furthermore, the present invention provides an agent for the treatment of patients being at the risk or suffering from a myocardial infarction, stroke or experienced a cardiovascular condition.

More specifically, the present invention relates to a method of determining the presence of an inflammatory disease and/or a cardiovascular or condition in a patient comprising assaying (i) a first sample taken from said patient for expression of Fibroblast Activation Protein (FAP), wherein expression or an increased expression of said FAP compared to a second, i.e. control sample typically obtained from a healthy subject is indicative for the disease or condition in said patient; and/or (ii) a first sample of a thrombus or plaque taken from said patient for expression of FAP, wherein an increased expression of FAP as compared to its expression in a second sample of a body fluid taken from said patient is indicative for the disease or condition. In a preferred embodiment, said body fluid sample is derived from blood.

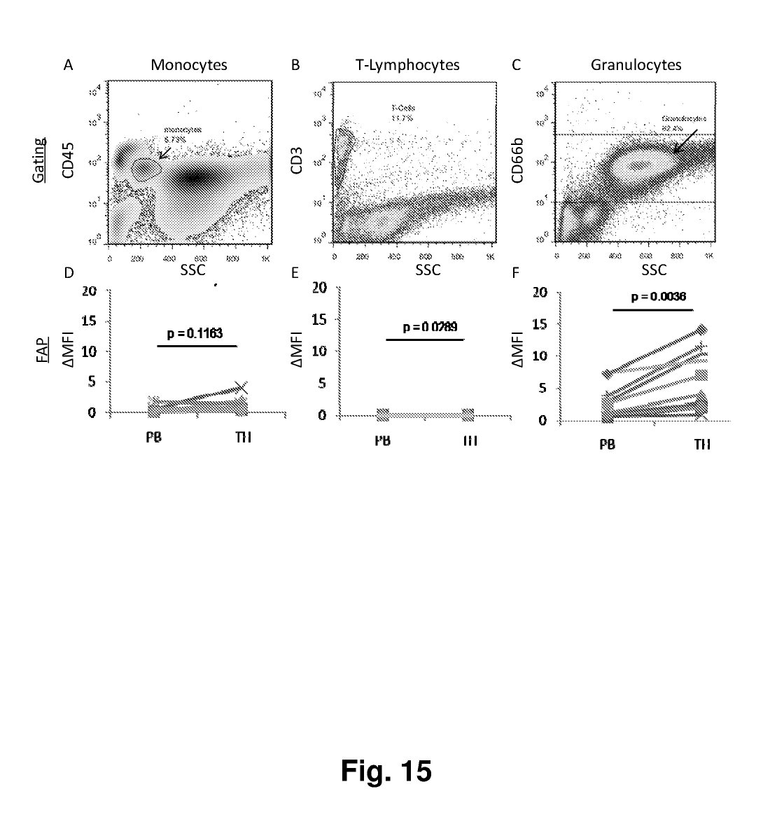

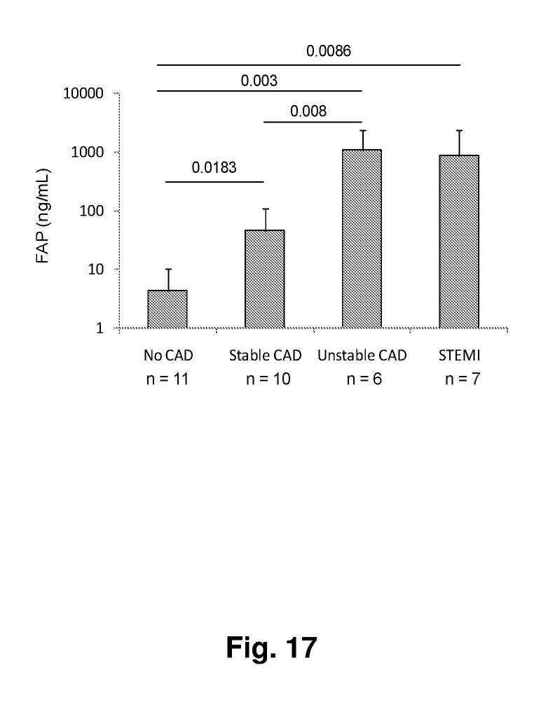

The versatility of the method of the present invention could further be proven by demonstrating that FAP is enhanced in the plasma of patients with acute myocardial infarction. For example, FAP expression in peripheral blood plasma harvested from patients experiencing an acute myocardial infarction and significant plaque (>30% occlusion of a coronary artery by coronary angiogram) is significantly increased compared to a sample from healthy patients with no risk factors or signs of significant atherosclerosis. In accordance with the experiments performed within the scope of the present invention it could be revealed that FAP plasma levels are enhanced in patient with acute coronary syndrome (ACS). In particular FAP levels are enhanced in patients with stable coronary artery disease (CAD). Further enhanced levels of FAP could be detected in patients with unstable angina and with ST-elevation myocardial disease (STEMI) compared to CAD patient exhibiting no CAD; see Example 13 and FIG. 17. Furthermore, it has been surprisingly found in accordance with the present invention that flow cytometry analysis revealed enhanced FAP expression in thrombus granulocytes, but not in the other analyzed cell populations of thrombi from the coronary artery aspirated from patients suffering from myocardial infarction as outlined in detail in Example 11 and corresponding FIG. 15. In the one embodiment of the first alternative (i) of the method of the present invention said first sample may be taken from coronary artery and said second sample from peripheral vein or the aortic sinus. In this embodiment, enhanced levels of FAP in the coronary circulation per se may be indicative for plaque vulnerability in accordance with the present invention.

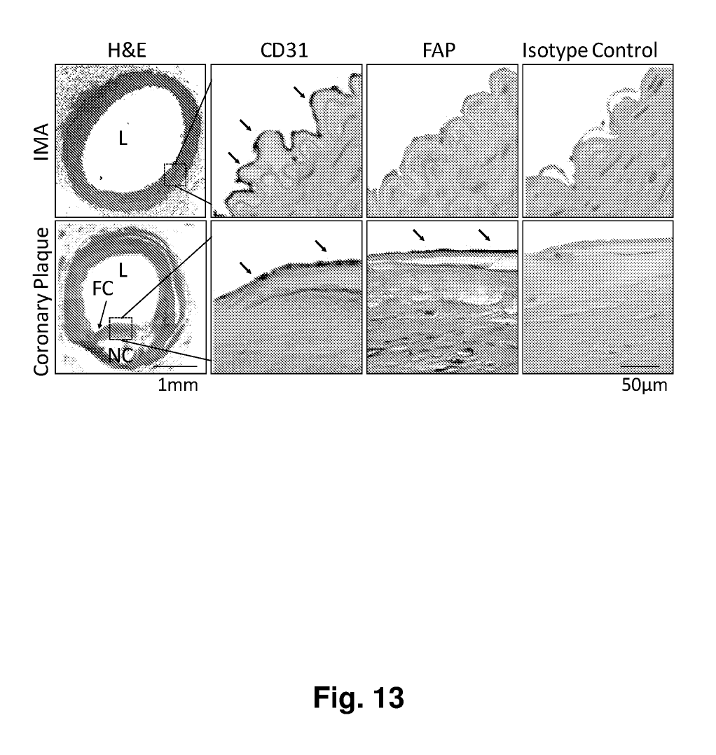

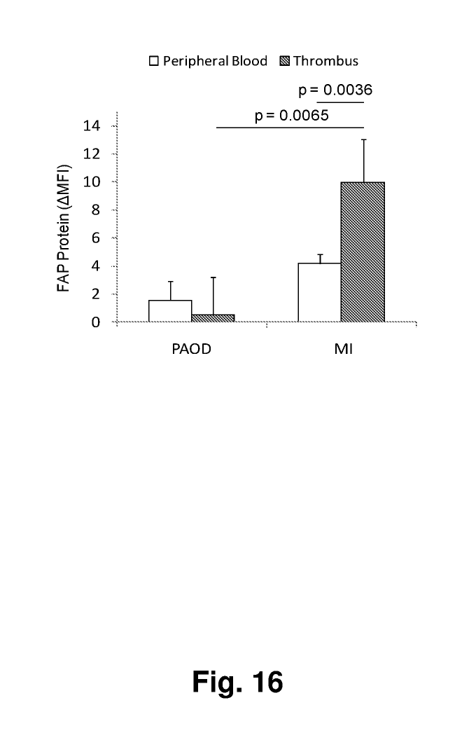

In addition, the experiments performed in accordance with the present invention revealed that FAP is expressed in occluding thrombi obtained from patients with myocardial infarction, see Example 12 and FIG. 16 as well as in rupture-prone human coronary arteries obtained from patients that died after myocardial infarction as described in Example 9 and FIG. 13.

In the one embodiment of the second alternative (ii) of the method of the present invention the first sample is preferably derived from a coronary thrombus, thrombus in iliac artery, carotid and/or coronary arteries, coronary stent derived thrombus, coronary thrombus leukocytes, coronary thrombus platelets, coronary atherosclerotic plaque endothelium, coronary atherosclerotic plaque fibrous cap, coronary atherosclerotic plaque lipid and/or necrotic core, coronary plaque adventitia, or carotid plaques. Leukocytes include monocytes, granulocytes including basophils, neutrophils and eosinophils, B-cells, T-cells including helper, cytotoxic, memory, regulatory, natural killer (NK), and gamma delta T-cell subsets.

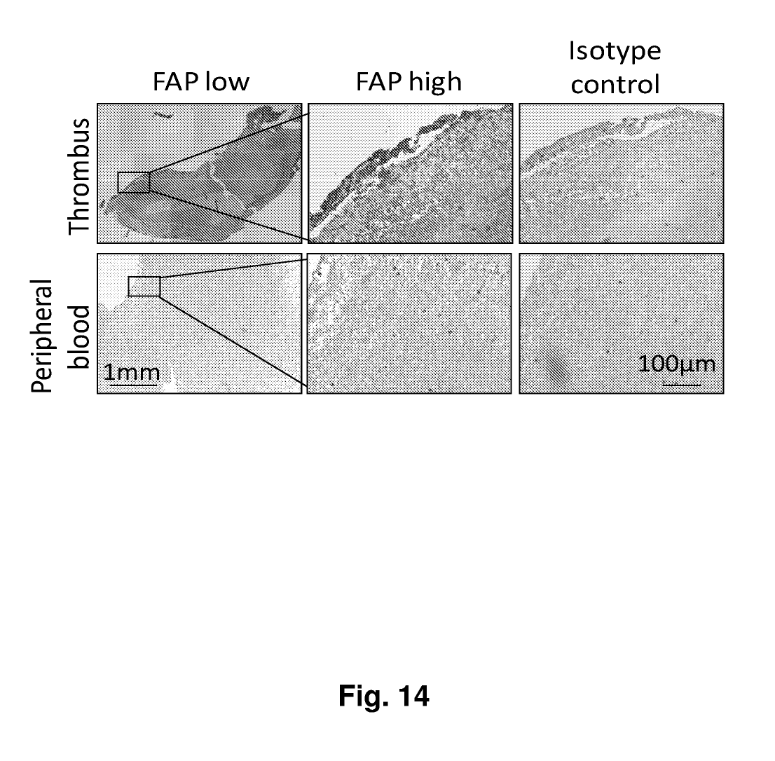

The second embodiment of the present invention is based on the observation that FAP expression is enhanced in coronary thrombi compared to peripheral blood in patients with an acute myocardial infarction, see FIG. 14 and Example 10. A further, more detailed flow cytometry analysis revealed that FAP is expressed by granulocytes (CD66b-positive) and not in monocytes (CD14-positive) in human coronary thrombi, see Example 11 and FIG. 15. Finally, the experiments of the present invention demonstrate enhanced FAP expression in acute but not chronic thrombosis when peripheral blood and occluding thrombi from patients suffering from ST-elevation myocardial infarction (STEMI) (coronary artery thrombi) and peripheral artery occlusive disease (PAOD) (femoral artery thrombi) samples are analyzed, see Example 12 and FIG. 16.

Previously, in international application WO 2006/010517 FAP has been associated with various diseases including cancer, respiratory diseases, dermatological diseases, metabolic diseases, inflammation, gastroenterological diseases, hematological diseases, muscle skeleton diseases, neurological diseases, urological diseases, endocrinological diseases and also cardiovascular diseases based on expression profiling only. However a credible link of FAP expression to inflammatory diseases and conditions has not been made. Moreover, no clinical data are disclosed at all which could provide evidence that any of the relative expression levels measured in the various human tissues is of significance, let alone decisive and reliable in the prediction of a disease or condition. Additionally, the role of FAP in coagulation and atherothrombosis has not been described. However, in order to conclude that a give target could potentially serve as a biomarker in clinical assessment its expression must be clearly attributable to a specific condition. Otherwise, the risk of determining false positive or other artifacts arise. Hence, measuring the expression level FAP in accordance with the teaching of WO 2006/010517 does not allow any conclusion as to the presence or risk of onset of a particular condition, if any.

Rather, the experiments performed in accordance with the present invention for the first time revealed that FAP is associated with and has a direct pathological role in atherosclerosis and atherothrombosis. Clinically, FAP is associated with the precise clinical event of plaque rupture and myocardial infarction. In particular, it has been found that FAP expression is enhanced in human aortic atheromata and in fibrous caps of rupture prone coronary arteries and correlates with inflammatory macrophage burden. It could be shown for the first time that FAP degrades collagen in atherosclerotic plaques. Moreover, it is shown for the first time that FAP accelerates blood coagulation, and results in a more stable clot formation. Therefore, FAP can be a marker for various diseases associated with inflammation of arteries and also a therapeutic target. In accordance with the present invention an inflammatory disease and/or a cardiovascular disease is selected from a group of atherosclerosis, rheumatoid arthritis, stroke or an acute coronary syndrome such as myocardial infarction heart attack, chronic liver disease, cerebral venous thrombosis, deep venous thrombosis or pulmonary embolism. Further examples of inflammatory diseases, e.g. cardiovascular diseases that can be treated, prevented or diagnosed with a method of the present invention include but not limited to thrombosis aneurism, heart failure, ischemic heart diseases, angina pectoris, sudden cardiac death, hypertensive heart disease, non-coronary vessel disease, such as atherosclerosis, small vessel disease, vascular dementia, nephropathy, hypertriglyceridemia, hypercholesterolemia, hyperlipidemia, xanthomatosis, asthma, hypertension, emphysema and chronic pulmonary disease; or a cardiovascular condition associated with an interventional procedure (procedure of vascular trauma) such as restenosis following angioplasty, placement of a stent tent, synthetic or neutral extension grafts. In a preferred embodiment, the condition is vulnerable atherosclerotic plaques or atherothrombosis.

The present invention discloses the use of the human Fibroblast Activation Protein (FAP) as a marker of the aforementioned diseases. Measurement of FAP can be used in the early detection of said disease or in the surveillance of patients who is suffering from the disease and undergoes therapeutic treatment. Thus, FAP expression may also be indicative for the success of the subject therapy, i.e. relapse or remission of for example thrombi and plaques, respectively.

The method of the present invention comprises assaying expression of FAP in various ways. For example, FAP protein, catalytic products or substrates of FAP, downstream and upstream signalling compounds as well as the mRNA level and gene expression of FAP can be analyzed in order to obtain data which are sufficient to determine the presence of FAP in a patient suffering or suspected to suffering from one of the above-mentioned disease. In a preferred embodiment of the present invention, the above-mentioned method comprises assaying said sample for FAP protein or mRNA. Thereby assaying for FAP protein expression indicates the presence of a disease and can also serve as control, when further candidate biomarkers are also assayed. In addition, the expression level of FAP can be used to determine the grade of progression and vulnerability of a atherosclerotic plaque, wherein high expression correlates with a high grade of vulnerability and wherein a low expression level of FAP give rise of the onset of a inflammatory disease, i.e. low grade of vulnerability. This information can be further used in the treatment or diagnosis of said patient.

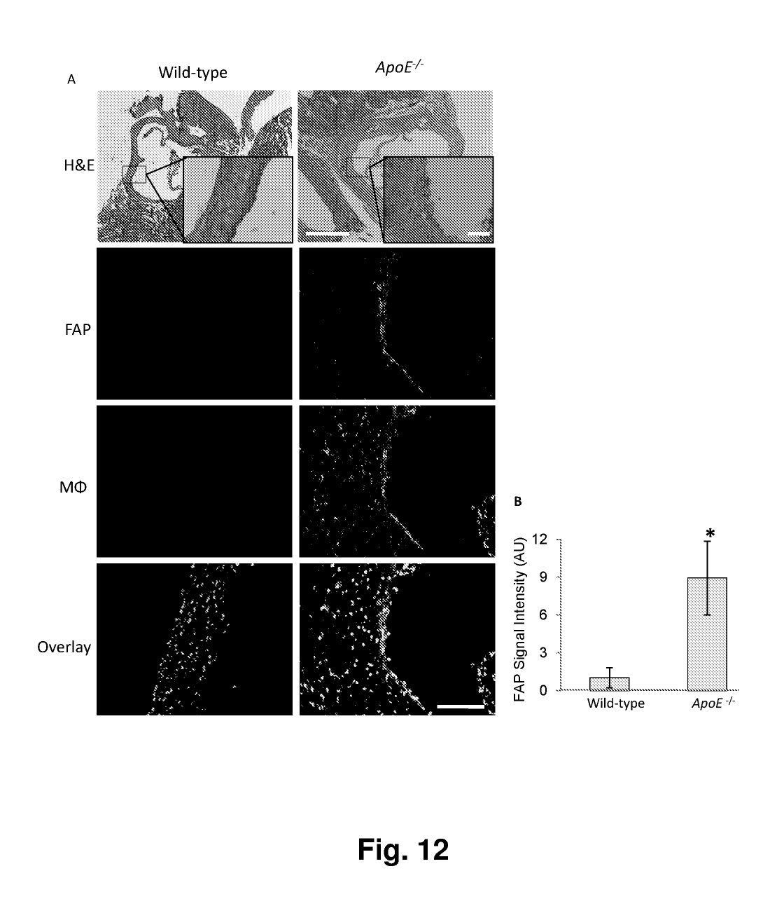

FAP expression is induced by inflammatory stimuli and enhanced in vulnerable human coronary plaques. Antibody-based inhibition of FAP's collagenolytic activity opens opportunities for targeted treatment and molecular imaging of vulnerable atherosclerotic plaques. In yet another aspect the present invention provides an in vivo system for studying therapeutic inhibition of FAP-mediated collagenolysis. As depicted in FIG. 12 and further explained in Example 6, FAP expression is enhanced in aortic atherosclerotic plaques of ApoE^{-/-} mice. The apolipoprotein E knockout mouse (ApoE^{-/-}) is a well-established model to study atherogenesis; see for further reading Steinberg N Engl J Med 320 (1989), 915-924 and Zhang Science 258 (1992), 468-471, the disclosure content of which is incorporated herein by reference. ApoE^{-/-} mice have decreased serum apolipoprotein E and exhibit lipid abnormalities and atherosclerosis even on a low-cholesterol diet. In accordance with the present invention the ApoE^{-/-} mice model can be used towards therapeutic inhibition of FAP-mediated collagenolysis and can contribute to identify the effects of FAP inhibiting on plaque stability by applying a blocking murine anti-FAP antibody in atherosclerotic mice. Moreover, as FAP is expressed in the atherosclerotic plaques of the ApoE^{-/-} mouse model this model could be crossed with an FAP double knockout mouse model (FAP^{-/-}) for use as a positive control to determine the maximal effect of FAP inhibition.

As the skilled artisan will appreciate, FAP has been identified as a maker which is useful in the assessment of inflammatory disease, e.g. cardiovascular disease. Identifying and assessing FAP can be achieved by various immunodiagnostic procedures or assays and may be used to reach a result comparable to the achievements of the present invention. In a preferred embodiment of the present invention, the assay is an immunoassay. Suitable immunoassays can be applied in either a direct or indirect format, like, for example, immunoprecipitation, particle immunoassays, radioimmunoassay (RIA), enzyme (EIA) immunoassay, fluorescent immunoassay (FIA) or chemiluminescent immunoassays. A variety of protocols for detecting and measuring the expression of FAP, using either polyclonal or monoclonal antibodies or fragments thereof specific for the polypeptide, is known in the art. In a preferred embodiment, said immunoassay comprises contacting said sample with a monoclonal or polyclonal antibody which binds specifically to FAP.

Antibodies against FAP are commercial available from different distributors, for example (Abcam, AbD Serotec, Abnova Corporation etc.). In a preferred embodiment, an antibody which is used for said assay is a monoclonal antibody. Suitable monoclonal antibodies can be generated in a mouse, rat, guinea pig, horse, pig, dog, cat or any other animal origin, preferably wherein the antibody according to the above-described method is a mouse anti-human antibody and/or a rabbit anti-human antibody Suitable antibodies and methods to detect FAP are also described in detail in Example II at page 6ff and Example IV at page 9ff in the international application WO 2007/111657, the respective disclosure content of which is herein incorporated by reference. This international application describes FAP expression in stationary cells associated with rheumatoid arthritis. In contrast to the present invention the international application makes use of a method to detect FAP exclusively in synovial tissues, i.e. synoviocytes. However, those kinds of stationary cells are preferably excluded in the method of the present invention wherein preferably a sample of a circulating body fluid sample such as blood is used, which is obtainable from the subject more conveniently and can also be more easily assayed. Accordingly, the present invention does not comprise the method for diagnosing rheumatoid arthritis via assaying myofibroblast-like synoviocytes for FAP as claimed and disclosed in international application WO 2007/111657.

An antibody which specifically binds to an epitope of FAP can be used diagnostically and therapeutically, as well as an immunohistochemical assays, such as Western blots, ELISA, radioimmunoassays, immunoprecipititations, cell fluorescence activated cytometry and/or cell sorting (FACS) magnetic activated cell sorting (MACS) or other immunochemical assays known in the art. General information and protocols are disclosed in Raem, Arnold M. Immunoassays. 1st ed., Munich; Heidelberg: Elsevier, Spektrum Akademischer Verlag., 2007; David Wild (Ed.): The Immunoassay Handbook. 3rd ed. Elsevier Science Publishing Company, Amsterdam, Boston, Oxford 2005. In a preferred embodiment, in the aforementioned method, said assay is an immunohistochemical assay.

Examples including direct or indirect, sandwich and cell-culture enzyme-linked immunoabsorbent assay (ELISA), a sandwich-type assay may be carried out with a capture and labelling antibody which is directed against the same epitope of FAP. In a typical forward sandwich assay, a first antibody having specificity for FAP is either covalently or passively bound to a solid or semi-solid support. The support is typically glass or a polymer, the most commonly used polymers being nitrocellulose, cellulose, polyacrylamide, nylon, polystyrene, polyvinyl chloride, polypropylene or mixture or derivatives of these. The solid supports may be in the form of tubes, beads, discs or microplates, or any other surface suitable for conducting an immunoassay. The binding processes are well-known in the art.

General formats and protocols for the conduct of various formats of ELISA are disclosed in the art and are known to those of skill in the field of diagnostics. For example, reference may be made to Chapter 11 of Ausubel (Ed) Current Protocols in Molecular Biology, 5^{th} ed., John Wiley & Sons, Inc, NY, 2002.

FACS and/or flow cytometry is a method of analyzing cell subpopulations, using automated equipment. It is widely used in medical labs and in biomedical and biochemical research, and it is discussed in various books and articles such as Flow Cytometry and Sorting, M. R. Melamed et al. (eds) Wiley and Liss, 1990 and in journals such as Cytometry and the American Journal of Clinical Pathology. FACS analysis can comprise flow cytometry as well as fluorescence activated cell sorting, wherein the conventional flow cytometry allows the analysis of fixed and permeabilized cells which are after analysis discarded. Fluorescence activated cell sorting is used to sort individual cells on the basis of optical properties, including fluorescence. It is used to screen large populations of cells in a relatively short period of time; thereby the sorted cells can be further processed or analyzed by suitable means. Other screening methods include Magnetic activated cell sorting MACS, as described in Gaines (1999) Biotechniques 26(4):683-688. Further reference to protocols means and methods can be obtained from the art Ormerod, M. G. (ed.) (2000) Flow Cytometry—A practical approach. 3rd ed. Oxford University Press, Oxford, UK ISBN 0199638241, Handbook of Flow Cytometry Methods by J. Paul Robinson ISBN 0471596345, et al. Current Protocols in Cytometry, Wiley-Liss Pub. In an preferred embodiment, the method according to the present invention comprising assaying a sample for FAP protein, wherein said assay comprises fluorescence activated cytometry (FACS), indirect ELISA, sandwich-ELISA or a cell culture ELISA.

However, the present invention is not limited to antibodies. Also agents which bind to FAP protein, for example interacting proteins or peptides or antibody derived molecules such as Fab, Fv or scFv fragments etc. in particular can be used. Antibodies or fragments thereof can be obtained by using methods which are described, e.g., in Harlow and Lane “Antibodies, A Laboratory Manual”, CSH Press, Cold Spring Harbor, 1988 or EP-A 0 451 216 and references cited therein. As a skilled artisan will appreciate their numerous method to measure the amount of specific binding agent FAP complex all described in detail in relevant text books (e.g. Tijssen P. or Diamandis, E. P. and Christopoulos, T. K., Immunoassay, Academic Press. Boston 1996).

Alternatively, in particular in the second alternative (ii) of the method of the present invention nucleic acid based means are used for assaying FAP, i.e. by determining FAP mRNA or protein expression. Suitable techniques for determining RNA expression levels in cells from a biological sample (e.g., Northern blot analysis, RT-PCR, in situ hybridization) are well known to those of skill in the art. For example, total cellular RNA can be purified from cells by homogenization in the presence of nucleic acid extraction buffer, followed by centrifugation. Nucleic acids are precipitated, and DNA is removed by treatment with DNase and precipitation. The RNA molecules are then separated by gel electrophoresis on agarose gels according to standard techniques, and transferred to nitrocellulose filters. The RNA is then immobilized on the filters by heating. Detection and quantification of specific RNA is accomplished using appropriately labeled DNA or RNA probes complementary to the RNA in question; see, for example, Molecular Cloning: A Laboratory Manual, J. Sambrook et al., eds., 2nd edition, Cold Spring Harbor Laboratory Press, 1989, Chapter 7, the entire disclosure of which is incorporated by reference.

Suitable probes for Northern blot hybridization can be produced from the nucleic acid sequences of FAP. Methods for preparation of labeled DNA and RNA probes, and the conditions for hybridization thereof to target nucleotide sequences, are described in Molecular Cloning: A Laboratory Manual, J. Sambrook et al., eds., 2nd edition, Cold Spring Harbor Laboratory Press, 1989, Chapters 10 and 11, the disclosures of which are incorporated herein by reference.

For example, the nucleic acid probe can be labeled with, e.g., a radionuclide, such as ³H, ³²P, ³³P, ¹⁴C, or ³⁵S; a heavy metal; or a ligand capable of functioning as a specific binding pair member for a labeled ligand (e.g., biotin, avidin or an antibody), a fluorescent molecule, a chemiluminescent molecule, an enzyme or the like. Probes can be labeled to high specific activity by either the nick translation method of Rigby et al., J. Mol. Biol. 113 (1977), 237-251 or by the random priming method of Feinberg et al., Anal. Biochem. 132 (1983), 6-13, the entire disclosures of which are incorporated herein by reference. The latter is the method of choice for synthesizing ³²P-labeled probes of high specific activity from single-stranded DNA or from RNA templates. For example, by replacing preexisting nucleotides with highly radioactive nucleotides according to the nick translation method, it is possible to prepare ³²P-labeled nucleic acid probes with a specific activity well in excess of 10⁸ cpm/microgram.

Autoradiographic detection of hybridization can then be performed by exposing hybridized filters to photographic film. Densitometric scanning of the photographic films exposed by the hybridized filters provides an accurate measurement of FAP mRNA gene transcript levels. Using another approach, FAP mRNA gene transcript levels can be quantified by computerized imaging systems, such the Molecular Dynamics 400-B 2D Phosphorimager available from Amersham Biosciences, Piscataway, N.J.

Where radionuclide labeling of DNA or RNA probes is not practical, the random-primer method can be used to incorporate an analogue, for example, the dTTP analogue 5-(N-(N-biotinyl-epsilon-aminocaproyl)-3-aminoallyl)deoxyuridine triphosphate, into the probe molecule. The biotinylated probe oligonucleotide can be detected by reaction with biotin-binding proteins, such as avidin, streptavidin, and antibodies (e.g., anti-biotin antibodies) coupled to fluorescent dyes or enzymes that produce color reactions.

In addition to Northern and other RNA hybridization techniques, determining the levels of RNA transcripts can be accomplished using the technique of in situ hybridization. This technique requires fewer cells than the Northern blotting technique, and involves depositing whole cells onto a microscope cover slip and probing the nucleic acid content of the cell with a solution containing radioactive or otherwise labeled nucleic acid (e.g., cDNA or RNA) probes. This technique is particularly well suited for analyzing tissue biopsy samples from subjects. The practice of the in situ hybridization technique is described in more detail in U.S. Pat. No. 5,427,916, the entire disclosure of which is incorporated herein by reference. Suitable probes for in situ hybridization of FAP mRNA can be produced from the nucleic acid sequences.

The relative number of FAP mRNA gene transcripts in cells can also be determined by reverse transcription of FAP mRNA gene transcripts, followed by amplification of the reverse-transcribed transcripts by polymerase chain reaction (RT-PCR). The levels of FAP mRNA gene transcripts can be quantified in comparison with an internal standard, for example, the level of mRNA from a “housekeeping” gene present in the same sample. A suitable “housekeeping” gene for use as an internal standard includes, e.g., 5S rRNA, U6 snRNA or tRNAs. The methods for quantitative RT-PCR and variations thereof are within the skill in the art.

In order to identify, diagnose or treat a disease or condition as mentioned above, the present invention also provides compositions which comprise small molecules that are able to inhibit, reduce, lower or retard an inflammatory disease by affecting the FAP molecule activity or its expression. Inhibition or reduction of FAP can be achieved by targeting the DNA or RNA or polypeptide of FAP by certain molecules or by destabilizing activators of FAP as well as activation of inhibitors of FAP. In one embodiment, the present invention relates to a composition comprising a compound capable of (i) detecting the presence or activity of FAP and/or (ii) inhibiting the activity of FAP or its expression for use in the diagnosis or treatment of a disease or condition as defined above.

Furthermore, the present invention relates to a composition for treating or diagnosing an inflammatory and/or a cardiovascular disease comprising the interacting molecule or compound as described above and optionally a pharmaceutical acceptable carrier. Put in other words, the composition in accordance with the present invention may be (i) a pharmaceutical composition and comprises a pharmaceutical acceptable carrier or (ii) a diagnostic composition and optionally comprises suitable means for the direct or indirect detection of FAP.

Regarding the second alternative (ii) of the composition of the present invention, i.e. diagnostic composition the compound may be any one of those described hereinabove for the determining FAP expression such as anti-FAP antibodies and FAP specific nucleic acid probes and primers. Preferably, the diagnostic composition is used in the method of the present invention as describe hereinbefore. Furthermore, anti-FAP antibodies and equivalent FAP binding molecules can be labeled (e.g., fluorescent, radioactive, enzyme, nuclear magnetic, heavy metal) and used to detect FAP in vivo in a manner similar to nuclear medicine imaging techniques to detect tissues, cells, or other material expressing FAP, for example thrombi and plaques. Targeting FAP and plaques with diagnostic imaging probes detectable by MM or PET would provide a biological marker for a more definitive premortem diagnosis of for example myocardial infarction a means for monitoring the efficacy of therapies.

Thus, in a further embodiment the present invention relates to the use of a FAP binding molecule such anti-FAP antibody or binding fragment thereof for the preparation of a composition for in vivo detection of or targeting a therapeutic and/or diagnostic agent to FAP, e.g in thrombi and plaques, suppressing thrombi and plaque formation or for extra-corporal extraction of FAP and pathological FAP expressing cells from body fluids.

Regarding the first alternative (i) of the composition of the present invention, i.e. pharmaceutical composition pharmaceutical acceptable carriers and administration routes can be taken from corresponding literature known to the person skilled in the art. The pharmaceutical composition of the present invention can be formulated according to the methods well known in the art; see for example Remington: Science and practice of pharmacy (2000) by the University of Science in Philadelphia, ISBN-0-683-306472. Examples of suitable pharmaceutical carriers are also known in the art and include phosphate buffers, saline solutions, water emulsions, emulsions, such as oil/water emulsions, various types of wetting agents, sterile solutions etc. Composition comprising such carriers can be formulated by well known conventional methods. The pharmaceutical compositions can be administered to the subject in a suitable dose. Administration of the suitable compositions may be effected by different ways. Examples include and administering a composition containing a pharmaceutically acceptable carrier via oral, intranasal, rectal, topical, intraperitoneal, intravenous, intramuscular, subcutaneous, subdermal, transdermal, interthectal and intercranial methods. Aerosol formulations such as nasal spray formulations include purified aqueous or other solutions of the active agent with preservative agents and isotonic agents. Such formulations are preferably adjusted to a pH and isotonic state compatible with nasal mucous membranes. Formulations for rectal or vaginal administration may be presented as a suppository with a suitable carrier. Further guidance regarding formulations that are suitable for various types of administration can be found in Remington's Pharmaceutical Sciences, Mace Publishing Company, Philadelphia, Pa., 17 th ed. (1985) and corresponding updates. For a brief review of methods for drug delivery see Langer, Science 249 (1990), 1527-1533.

As is well known in the medical arts, dosages for any one patient depends upon may factors, including the patient's size, body surface area, age, the particular compound to be administered, sex, time and route of administration, general health, and other drugs being administered concurrently. In addition, co-administration or sequential administration of other agents may be desirable. A therapeutically effective dose or amount refers to that amount of the active ingredient sufficient to ameliorate the symptoms or condition. Therapeutic efficacy and toxicity of such compounds can be determined by standard pharmaceutical procedures in cell cultures or experimental animals, e.g., ED50 (the dose therapeutically effective in 50% of the population) and LD50 (the dose lethal to 50% of the population). The dose ratio between therapeutic and toxic effects is the therapeutic index, and it can be expressed as the ratio, LD50/ED50.

The compound in the composition of the present invention may directly inhibit the activity, expression or processing of FAP. For example, the compound can interact with, e.g., bind to, a FAP protein or fragment thereof and block or reduce the FAP activity, e.g. collagenase activity and (blood) clotting activity. In a further preferred embodiment, the composition comprises a compound with an activity, wherein said activity is an enzymatic activity. In still another embodiment, the composition can block the processing of FAP, e.g. the compound can inhibit one or more of: the conversion of FAP from a precursor to active form, or the release or secretion of active or latent forms of FAP. Alternatively, the composition or compound can indirectly inhibit FAP by inhibiting the activity, e.g. the enzymatic activity or expression of: an upstream factor activator or of tumor necrosis factor alpha (TNF-α), or transforming growth factor beta (TGF-β), an enzyme involved in the conversion of FAP from latent to active form or a downstream FAP activated target; or can increase activity or expression of an FAP inhibitor, or a downstream FAP inhibitor target.

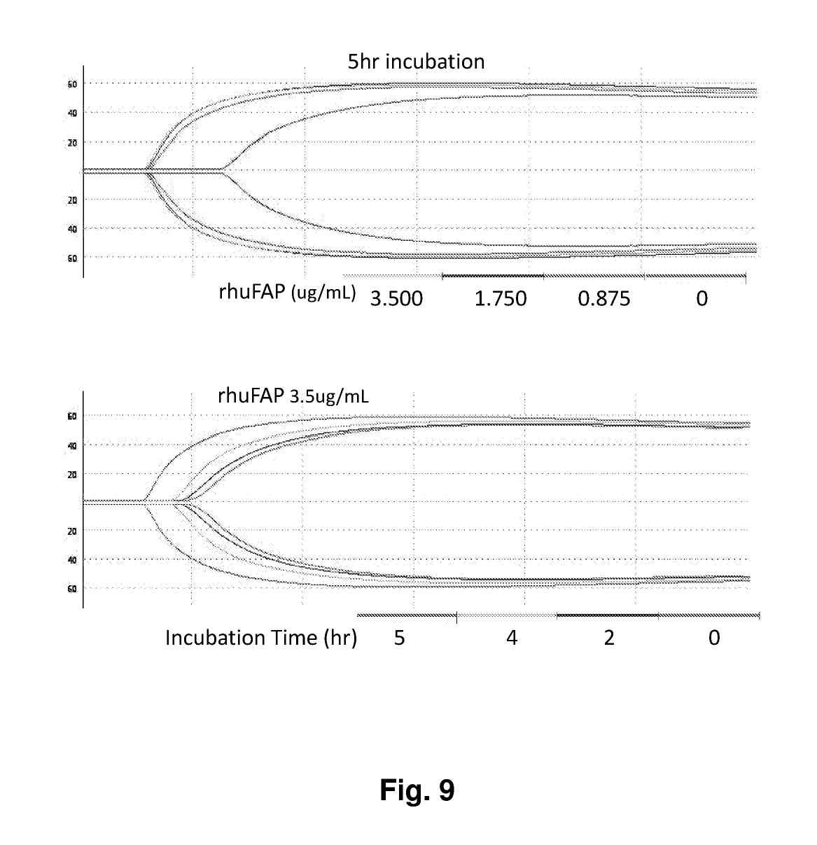

A further aspect of the present invention is based on the surprising finding that FAP accelerates the rate of human blood clotting as shown in the Example 8 and in FIGS. 9 to 11. Blood clotting or blood clots are natural parts of the healing process in a body. Blood thickness around an injured area forms a protective barrier and eventual dissolves on its own. However, clots become dangerous when they interfere with the circularity system. Thrombi (blood clots) are leading cause of death and disability. Blood clots are responsible for health problems, including stroke, heart attack, pulmonary embolism and complications of cancer. In a preferred embodiment the composition in accordance with the present invention is suitable for use in inhibiting blood clotting.

In another embodiment of the present invention, the compound in the pharmaceutical composition can block or reduce expression of FAP, e.g. by reducing transcription or translation of FAP mRNA, or reducing the stability of FAP mRNA or protein. Thereby, the compound is an inhibitor of the expression or translation of an FAP nucleic acid such as a double-stranded RNA (dsRNA) molecule, microRNA (miRNA), an antisense molecule, a ribozyme, a triple-helix molecule, or any combination thereof. In one preferred embodiment of the present invention the said compound is capable of binding to FAP or its encoding nucleic acid molecule.

In a further preferred embodiment according to the present invention, the compound is an agent which is a small molecule, e.g. a chemical agent, a small organic molecule, e.g. a product or a combinatory of natural product library, a polypeptide, e.g. an antibody such as an FAP specific antibody, a peptide, a peptide fragment, e.g. a substrate fragment such as of collagen I fragment, a peptidometic or a modulator. Preferably, the compound is an FAP specific antibody, means and methods to generate such antibodies are described supra. In a further preferred embodiment according to the present invention said compound is an antibody which specifically binds to FAP such as a polyclonal antibody, a monoclonal antibody, a human antibody, humanized antibody, a chimeric antibody, a recombinant antibody and a synthetic antibody. Preferably, said monoclonal antibody is a human antibody. Suitable methods and guidance for the generation of human antibodies are described in detail in the international application WO 2008/081008 the disclosure content of which is incorporated herein by reference. Antibody as used herein includes intact immunoglobulin molecules, as well as fragments thereof, such as Fab, F(ab′)₂, and Fv, which are capable of binding an epitope of FAP.

The compounds can also be antagonists or agonists of FAP polypeptide and can be compounds that exert their effect on the FAP activity via the enzymatic activity, expression, post-translational modifications or by other means. Agonists of FAP are molecules which, when bound to FAP increase or prolong the activity of FAP. Agonists of FAP include proteins, nucleic acid, carbohydrates, small molecules or any other molecules which activate FAP.

In one embodiment, the present invention relates to a composition, wherein said compound is a FAP antagonist. Antagonists of FAP are for example molecules which, when bound to FAP, decrease the amount of the duration of the activity of FAP. Inhibitors or antagonists are capable in inhibiting the activity of FAP polypeptide, mRNA or DNA level or its expression refers to a change in the activity of FAP, by decreasing in the enzymatic activity or by affecting transcription or translation of FAP, binding characteristics or any other biological, functional or immunological properties of FAP. Antagonists may be peptides, proteins, nucleic acid, carbohydrates, antibodies, small organic compounds, peptide mimics, aptamers or PNAs (Milner, Nature Medicine 1 (1995), 879-880; Hupp, Cell 83 (1995), 237-245; Gibbs, Cell 79 (1994), 193-198; Gold, Ann. Rev. Biochem. 64 (1995), 736-797). For the preparation and application of such above-mentioned compounds, the person skilled in the art can use the methods known in the art, for example those referred to in the cited literature. Preferred according to the present invention is a composition, wherein said antagonist is a peptide or peptide analog. Suitable methods to obtain peptides or peptide analogs to be used in inhibiting FAP activity are known in the art and described in detail, e.g., in the international application WO2006/125227, the disclosure content with respect to the FAP inhibitors of which is enclosed herein by reference. This application describes a method, wherein the use of N-blocked peptide proline boronate compounds are used to inhibit FAP protein in order to be used in the treatment of hyperproliferative disorders such as cancer.

Further means and methods for providing a pharmaceutical or diagnostic composition in accordance with the present invention are known to the person skilled in the art and are described for example in international application WO 2006/010517; see also supra, the disclosure content of which is incorporated herein by reference.

In a preferred embodiment, the pharmaceutical composition of the present invention is used for the treatment of a patient who has been diagnosed in accordance with the method of the present invention as described herein. In one embodiment, the compound or the pharmaceutical composition of the present invention is designed to be administered to a subject in combination with an inflammatory agent that is being used to treat a related disorder, e.g. atherosclerosis, myocardial infarction, stroke, thrombosis, heart failure, angina pectoris, sudden cardiac death or a cardiovascular condition. Preferred cardiovascular disorders include atherosclerosis, myocardial infarction, acute syndromes, aneurysm and stroke.

In a preferred embodiment, the subject to be treated and/or diagnosed is a human suffering from or at a risk of an FAP mediated/associated disorder or disease, e.g. an inflammatory disease and/or a cardiovascular disease as described herein. Most preferably, the subject is a human suffering from, or is at risk of suffering from atherosclerosis. For example, the subject is a human with early intermediate or advanced atherosclerosis or is at a risk of rupture of an atherosclerotic plaque. The method includes administering to a subject an agent that inhibits the activity, expression, translation or processing of FAP, e.g. a compound as described herein, in an amount effective to reduce or inhibit FAP and/or FAP-mediated blood clotting. In a preferred embodiment, the method further includes evaluating FAP, nucleic acid or protein expression level or activity in a subject before or after administration step. For example, a subject, e.g. a patient at risk of atherosclerotic plaque rupture, can be evaluated before or after the compound is administered. If the subject has a level of FAP above predetermined level, therapy can begin or to be continued.

The present invention also relates to the prevention or treatment of thromboembolic diseases, preferably of a coagulation disorder, e.g. blood clotting disorders, various blood diseases with homeostatic or clotting disorders. Typical examples of a blood clotting disorder include coagulation disorders like hemophilia A, hemophilia B, Willebrand disease, disseminated intravascular coagulation (DIC), severe liver disease, and Vitamin K deficiency and any other causes. Experiments performed in accordance with the present invention revealed that FAP protein can enhance the blood clotting, as shown by thrombelastographic analysis in Example 8 and FIGS. 9 to 11. In particular, representative ROTEM readouts from a NATEM assay treated with increasing concentrations of recombinant human FAP over increasing incubation periods are shown and reveal that FAP accelerates the rate of human blood clotting. ROTEM® stands for rotation thromboelastometry and is an enhancement of classical thromboelastography. The thrombelastography (ROTEM®) is for a person skilled in the art a well known technique which gives a graphic representation of clot formation and subsequent lysis. Blood is incubated in a heated cup. Within the cup is suspended a pin connected to a detector system, which is an optical detector. The cup and pin are oscillated relative to each other through an angle of 4_{—}45¢. The movement is initiated from the pin. As fibrin forms between the cup and the pin the transmitted rotation is detected at the pin and a trace is generated. The loss of the elasticity when the sample clots leads to a change in the rotation of the axis. These obtained data are then visualized in a thromboelastogram, for further reading see Luddington et al., Clin. Lab. Haem. 27 (2005), 81-90, the disclosure content of which is incorporated herein by reference. In a preferred embodiment the present invention relates to the use of ROTEM/NATEM as described herein and in Example 8 for screening of a potential recombinant coagulation factor or inhibitor thereof. Advantageously, the present invention relates to the use of ROTEM/NATEM for testing the therapeutic effect and/or efficiency of FAP blocking agents in a patient.

Here, the present invention provides for the first time the use of the ROTEM/NATEM technique to examine pro-coagulant activity of a recombinant protein, i.e. FAP. In accordance with the present invention this approach can be used to test the therapeutic effect of blocking FAP (to slow coagulation) and/or the therapeutic effect of adding FAP (to accelerate blood coagulation) in human blood. In another embodiment of the present invention, this method can be used to test the efficacy of FAP blocking agents, and also to determine effective doses of such blocking agents. Thus, in a further aspect the present invention relates to the use of the above-mentioned ROTEM/NATEM system for identifying and analyzing the coagulant activity of recombinant proteins or their inhibitors.

As mentioned, the present invention also provides a method to diagnose coagulation disorders, wherein the use of FAP as a reagent for enhancing blood clotting in vivo can be used. In a preferred embodiment, the present invention relates to the pharmaceutical composition comprising FAP for the use and the prevention or treatment of a coagulation disorder. In a preferred embodiment the coagulation disorder is hemophilia. Therefore, according to the present invention FAP could used as a reagent for enhancing blood clotting in vitro. Accordingly, the invention provides an in vitro method for the diagnosis of blood coagulation disorders in a human individual or for the determination of the risk for a human individual to acquire said blood conjugation disorder.

The above disclosure generally describes the present invention. Several documents are cited throughout the text of this specification. Full bibliographic citations may be found at the end of the specification immediately preceding the claims. The contents of all cited references (including literature references, issued patents, published patent applications as cited throughout this application and manufacturer's specifications, instructions, etc) are hereby expressly incorporated by reference; however, there is no admission that any document cited is indeed prior art as to the present invention.

A more complete understanding can be obtained by reference to the following detailed description and experiments which is provided herein for purposes of illustration only and is not intended to limit the scope of the invention.

### EXAMPLES

The examples which follow further illustrate the invention, but should not be construed to limit the scope of the invention in any way. Detailed descriptions of conventional meth-ods, such as those employed herein can be found in the cited literature; see also “The Merck Manual of Diagnosis and Therapy” Seventeenth Ed. ed by Beers and Berkow (Merck & Co., Inc. 2003).

The practice of the present invention will employ, unless otherwise indicated, conventional techniques of cell biology, cell culture, molecular biology, transgenic biology, microbiology, recombinant DNA, and immunology, which are within the skill of the art.

Methods in molecular genetics and genetic engineering are described generally in the current editions of Molecular Cloning: A Laboratory Manual, (Sambrook et al., (1989) Molecular Cloning: A Laboratory Manual, 2nd ed., Cold Spring Harbor Laboratory Press); DNA Cloning, Volumes I and II (Glover ed., 1985); Oligonucleotide Synthesis (Gait ed., 1984); Nucleic Acid Hybridization (Hames and Higgins eds. 1984); Transcription And Translation (Hames and Higgins eds. 1984); Culture Of Animal Cells (Freshney and Alan, Liss, Inc., 1987); Gene Transfer Vectors for Mammalian Cells (Miller and Calos, eds.); Current Protocols in Molecular Biology and Short Protocols in Molecular Biology, 3rd ed. (Ausubel et al., eds.); and Recombinant DNA Methodology (Wu, ed., Academic Press). Gene Transfer Vectors For Mammalian Cells (Miller and Calos, eds., 1987, Cold Spring Harbor Laboratory); Methods In Enzymology, Vols. 154 and 155 (Wu et al., eds.); Immobilized Cells And Enzymes (IRL Press, 1986); Perbal, A Practical Guide To Molecular Cloning (1984); the treatise, Methods In Enzymology (Academic Press, Inc., N.Y.); Immunochemical Methods In Cell And Molecular Biology (Mayer and Walker, eds., Academic Press, London, 1987); Handbook Of Experimental Immunology, Volumes I-IV (Weir and Blackwell, eds., 1986). Reagents, cloning vectors, and kits for genetic manipulation referred to in this disclosure are available from commercial vendors such as BioRad, Stratagene, Invitrogen, and Clontech. General techniques in cell culture and media collection are outlined in Large Scale Mammalian Cell Culture (Hu et al., Curr. Opin. Biotechnol. 8 (1997), 148); Serum-free Media (Kitano, Biotechnology 17 (1991), 73); Large Scale Mammalian Cell Culture (Curr. Opin. Biotechnol. 2 (1991), 375); and Suspension Culture of Mammalian Cells (Birch et al., Bioprocess Technol. 19 (1990), 251); Extracting information from cDNA arrays, Herzel et al., CHAOS 11, (2001), 98-107. Materials and techniques for design and construction of labeled antibodies and other agents for use in cytometry are known in the art and described for example, in Bailey et al. (2002) Biotech. Bioeng. 80(6); 670-676; Carroll and Al-Rubeai (2004) Expt. Opin. Biol. Therapy 4:1821-1829; Yoshikawa et al. (2001) Biotech. Bioeng. 74:435-442; Meng et al. (2000) Gene 242:201-207; Borth et al. (2001) Biotechnol. Bioeng. 71 (4):266-273; Zeyda et al. (1999) Biotechnol. Prog. 15:953-957; Klucher et al. (1997) Nucleic Acids Res. 25(23):4853-4860; and Brezinsky et al. (2003) J. Imumunol. Methods 277:141-155.

### Experimental Procedures

### Characterization of Atherosclerotic Plaques

Ascending aortic plaque biopsies are obtained from patients undergoing operations for aortic stenosis or aortic valve replacements. Aortic plaques are sectioned and graded according the American Heart Association (AHA) criteria^{20,21} using Movat pentachrome, Oil-Red-O, anti-CD68, and von Kossa staining. Coronary arteries are obtained from patients that died after an acute myocardial infarction and embedded in paraffin for sectioning. Vulnerable coronary plaques are characterized by Masson staining against collagen in tissue sections. Fibrous caps are identified as the collagen-rich tissue separating the lumen and the necrotic core.² Plaques with a minimum fibrous cap thickness of less than 50 μm are classified as unstable, whereas plaques with a fibrous cap thickness of greater than 65 μm are classified as stable.²

### Immunofluorescence and Immunohistochemistry

Cross sections from human ascending aortae (10 μm thickness) and paraffin-embedded sections of coronary plaques (4 μm thickness) are mounted on glass slides. Tissue sections are labeled against FAP and cell specific markers with purchased antibodies directed against CD68, von Willebrand factor, alpha smooth muscle actin, or type I collagen and visualization with either fluorescently labeled secondary antibodies or biotin labeled secondaries for immunostaining using an ABC staining kit for diaminobenzidine (Vector Labs, Burlingame, Calif.).

### Immunofluorescence

Paraffin-embedded coronary sections are labeled with A246 and subsequently by Cy5-labeled secondary goat anti-mouse IgG (115-175-146; Jackson ImmunoResearch). For detailed methods of immunofluorescence, FAP colocalization with macrophages, endothelial cells, and smooth muscle cells are determined using a rabbit antibodies directed against CD68 (SC-9139; Santa Cruz, Santa Cruz, Calif.), von Willebrand factor (vWF; F3520; Sigma-Aldrich, Carlsbad, Calif.), alpha-smooth muscle actin (αSMA; Ab5694; Abcam), or type I collagen (Ab292; Abcam); subsequent fluorescent labeling is performed using a Cy3-labeled anti-rabbit IgG (111-165-144; Jackson ImmunoResearch) in three adjacent sections of each biopsy specimen. DAPI (D9542; Sigma-Aldrich) is used for fluorescent counterstaining of nuclei. Isotype control antibodies are used to address antigen-binding specificity. Stained samples are cover-slipped with Tris-buffered glycerol (a 3:7 mixture of 0.1 M Tris-HCl at pH 9.5 and glycerol supplemented with 50 mg/mL n-propyl-gallate).

### Immunohistochemistry

Antigen retrieval in paraffin-embedded sections of coronary plaques is performed after 20 min incubation in 95° C. retrieval buffer (2 mM Sodium Citrate, pH 7.6). FAP is stained using a rabbit polyclonal antibody raised against the catalytic insert of FAP (A246; Ab28246; Abcam, Cambridge, Mass.) and a rabbit isotype control (Ab37415; Abcam). Cryosections of aortic plaques are fixed in ice cold acetone for 5 min and stained a mouse monoclonal against FAP (F19, Provided by Sloan-Kettering Institute, New York, N.Y.) with the appropriate mouse isotype control (401401; BioLegend, San Diego, Calif.). Primary antibodies are labeled with biotin-labeled goat anti-mouse (115-066-003; Jackson ImmunoResearch, West Grove, Pa.) and biotin-labeled goat anti-rabbit (111-066-003; Jackson ImmunoResearch) and are stained using an ABC staining kit for diaminobenzidine (Vector Labs, Burlingame, Calif.)

### Image Analysis

For low power imaging at spatial resolutions above 1 μm/pixel, a fluorescent microscope (DM60000B; Leica, Wetzlar, Germany) equipped with a fluorescent camera (DFC350 FX; Leica) are used.

Colocalization analyses are performed at higher magnifications using a multichannel confocal microscope (TCS SP2, Leica) on a single optical plane. Detailed image analysis methods are described in the Quantitative Image Analysis section immediately below.

### Quantitative Image Analysis

Single-channel fluorescent images (nine images per section in three adjacent sections) of aortic tissue sections are taken at constant camera settings in tagged image file format (TIFF) at a binary pixel intensity between 0-255. In each group, additional adjacent tissue sections are stained with isotype control antibodies to determine the background threshold. The background intensity threshold for each channel is set at the intensity under which 95% of the pixels emitted in control staining. Pixels below the background intensity threshold are excluded from quantification.²⁵ The remaining pixels are summed and divided by the total number of pixels to calculate the mean pixel intensity and the positive pixels were summed to calculate positive area using image analysis software by Matlab (Mathworks, Novi, Mich.).²⁶

Quantitative colocalization analyses are performed using a confocal microscope at high resolution on a single plane. Two TIFF images are captured from distinct fluorescent channels on each tissue section, and background signal subtracted as previously described. The colocalization coefficients are calculated as the sum of the FAP positive pixels which colocalized with pixels positive for cell specific markers.^{27,28}

### Cells

Human aortic endothelial cells (HAEC) are isolated from biopsies of ascending aortae without macroscopic lesions obtained from patients undergoing operations for valve repair, human aortic smooth muscle cells are purchased (HASMC; Promocell) and peripheral blood monocytes, macrophages, and foam cells are isolated from peripheral blood of healthy probands. For detailed methods of cell isolation, expansion, and purity validation please see below.

### Cells Isolation, Expansion, and Validation

For endothelial cell isolation, aortic lumens are washed with PBS and incubated in DMEM containing collagenase type 2 (350 U/mL; Worthington, Lakewood, N.J.) for 30 min, and agitated gently to dissociate the endothelium from the vessel wall. Endothelial cells are further purified by magnetic bead separation against CD34 (130-046-702; Miltenyi Biotec, Gladbach, Germany).

HAEC are expanded in endothelial cell growth medium (EGM2; Gibco) and characterized by FACS analysis for vWF expression (>98%). To isolate phagocytic monocytes, 50 mL of peripheral venous blood is collected from healthy probands in EDTA collection tubes, diluted 2× in Hank's buffered salt solution and spun on a Ficoll gradient (20 min, 400 G, 24° C.). Monocytes are selected from the buffy coat by magnetic bead sorting for CD14 (130-050-201; Miltenyi Biotec), to yield a final purity over 94% (FACS against CD64). Monocytes are differentiated into macrophages in polystyrene six well plates over seven days in RPMI-1640 (Gibco) containing 10% heat-inactivated, low-endotoxin fetal bovine serum (Gibco) and 50 nM recombinant human macrophage colony stimulating factor (AF-300-03; Peprotech, Rocky Hill, N.J.), replacing the media every 48 hr. Macrophage differentiation is validated using anti-CD68 FACS analysis. FAP is measured in all cell types using F19 in unfixed cells and a Cy5-conjugated secondary antibody for FACS analyses, and the appropriate murine IgG isotype control antibody. Foam cells are generated by stimulating macrophages with 100 μg/mL of oxidized low-density lipoprotein (BT-910; BioConcept, Allschwil, Switzerland) for 48 hr in Serum Free Macrophage Medium (SFM; Gibco). Lipid uptake is validated by Oil-red-0 staining (O0624; Sigma-Aldrich).

### Aortic and Coronary Specimens

Ascending aortic plaque biopsies are obtained from patients undergoing operations for aortic stenosis. Plaque-free aortic biopsies are obtained from patients undergoing operations to repair valve defects. These samples are placed directly into sterile Dulbecco's Modified Eagle Medium (DMEM; Gibco, Carlsbad, Calif.), pre-chilled to 4° C., and transferred on ice to the laboratory and frozen in Optimal Cutting Temperature Compound (Tissue-Tek, Torrance, Calif.) for tissue zymography.

Coronary arteries are obtained from patients that died after an acute myocardial infarction, placed in 4% paraformaldehyde solution for 24 hr.

### FAP Induction Assays

Quiescent HASMCs are treated with starvation media supplemented with 3, 5, 10, 20, 40% macrophage-conditioned SFM for 48 hr. To determine the effects of TNFα on FAP expression, quiescent HASMCs are treated with starvation media supplemented with 20% macrophage-conditioned SFM and a TNFα neutralizing antibody (Ab6671; Abcam) or an IgG isotype control (Ab27478; Abcam) antibody. Recombinant human TNFα (300-01A; Peprotech) is used to induce FAP expression in quiescent HASMC in a dose- and time-dependant manner. All FAP levels are quantified by cell membrane ELISA detailed below. Peripheral blood derived macrophages are incubated for 48 hr in SFM (2 mL/well in a 6 well plate) and the supernatant is sterile filtered, aliquoted, and frozen at −80° C. Purchased human aortic smooth muscle cells (HASMC; Promocell) are validated by FACS analyses for αSMA (purity>96%), and plated at passage 4 into a 96-well black cell culture test plate at a density of 5×10⁴ cells/cm² in DMEM (10938; Gibco) supplemented with 10% Fetal Calf Serum (3302-P250302; PAN Biotech, Aidenbach, Germany) and allowed to attach for 24 hr before rendered quiescent overnight in Advanced DMEM (12491-015; Invitrogen, Carlsbad, Calif.) with 1% bovine serum albumin (starvation medium). Quiescent HASMCs are treated with starvation media supplemented with 3, 5, 10, 20, 40% macrophage-conditioned SFM for 48 hr. To determine the effects of TNFα on FAP expression, quiescent HASMCs are treated with starvation media supplemented with 20% macrophage-conditioned SFM and a TNFα neutralizing antibody (Ab6671; Abcam) or an IgG isotype control (Ab27478; Abcam) antibody. Recombinant human TNFα (300-01A; Peprotech) is used to induce FAP expression in quiescent HASMC in a dose- and time-dependant manner. For quantifications, cells are washed with PBS, fixed in 4% formalin for 30 min, and labeled against FAP with F19. Cultures were enzymatically labeled using a Horseradish Peroxidase ELISA kit (Anaspec, Fremont, Calif.) and the enzymatic product quantified with a fluorescent plate reader. Background values are subtracted, and all values are normalized to untreated/unconditioned control groups.

### Zymography

Direct-quenched porcine gelatin (DQ gelatin; D12054; Invitrogen) is diluted to a final concentration of 100 μg/mL in reaction buffer (0.5 M Tris-HCl, 1.5 M NaCl, 50 mM CaCl₂, and 2 mM sodium azide at pH 7.6) and 0, 10, 20, 40 nM of recombinant human FAP in a black 96-well plate. Cleaved gelatin was quantified, at 0.5, 8, and 24 hr, and background fluorescence subtracted. A246 raised against FAP's catalytic insert and a matching isotype control IgG (Ab27478; Abcam) are added to 20 mM FAP and 100 μg/mL DQ gelatin in reaction buffer and analyzed after 24 hr, to determine blocking efficacy.

A246 is a rabbit polyclonal raised against an FAP-specific peptide, immunogen affinity purified, and recognizes fibroblast activation protein specifically, but not other dipeptidyl peptidases family members (Abcam; Ab28246). Confluent HASMC at passage 4 were treated with A246 or an antibody control for 30 min, washed with PBS, and then placed under 100 μL of 100 μg/mL DQ gelatin in reaction solution for 4 hr before fluorescence analysis with a plate reader. In situ zymography is performed on (5 μm) cryosections of human aortic atherosclerotic plaques which is stained against FAP using non-inhibitory F19 and Cy5 labeled secondary antibody, and then are treated with A246 or an isotype control at 50 nM concentration overnight.¹⁷ Treated and untreated sections are then mounted in warm 1% Agarose in PBS with 10% direct quenched type I collagen from bovine skin (D12060; Invitrogen) and are imaged after 2 hr at 37° C. by confocal microscopy. For quantification, background signals are subtracted from isotype control images and pixels which are positive for both FAP cleaved Type I collagen are quantified as the average of nine images from three adjacent sections per biopsy.

Direct-quenched porcine gelatin is added to increasing concentrations of recombinant human FAP in a black 96-well plate. Inhibition of FAP is perform with A246; a rabbit polyclonal raised against an FAP-specific peptide, that is immunogen affinity purified, and recognizes FAP specifically, but not other dipeptidyl peptidases family members (Abcam; Ab28246). Cleaved gelatin is quantified by a fluorescent plate reader, at 0.5, 8, and 24 hr, and background fluorescence subtracted.

In situ zymography is performed on (5 μm) cryosections of human aortic atherosclerotic plaques which is stained against FAP using non-inhibitory F19 and Cy5 labeled secondary antibody, and then treated with A246 or an isotype control at 50 nM concentration overnight. Treated and untreated sections are then mounted in warm 1% Agarose in PBS with 10% direct quenched type I collagen from bovine skin (D12060; Invitrogen) and imaged after 2 hr at 37° C. by confocal microscopy, and quantified as previously described.

### Statistical Analyses

Histological and cell culture results are compared using a one-way ANOVA and associations calculated by Pearson's correlation coefficient. Student's T-test is used for comparisons of zymography. All statistical analyses are performed using MatLab (Version, R2007b). Data are presented as mean±SD. Significance is accepted at the level of p<0.05.

### Example 1

### FAP is Expressed by Smooth Muscle Cells, but not Macrophages in Advanced Human Aortic Plaques

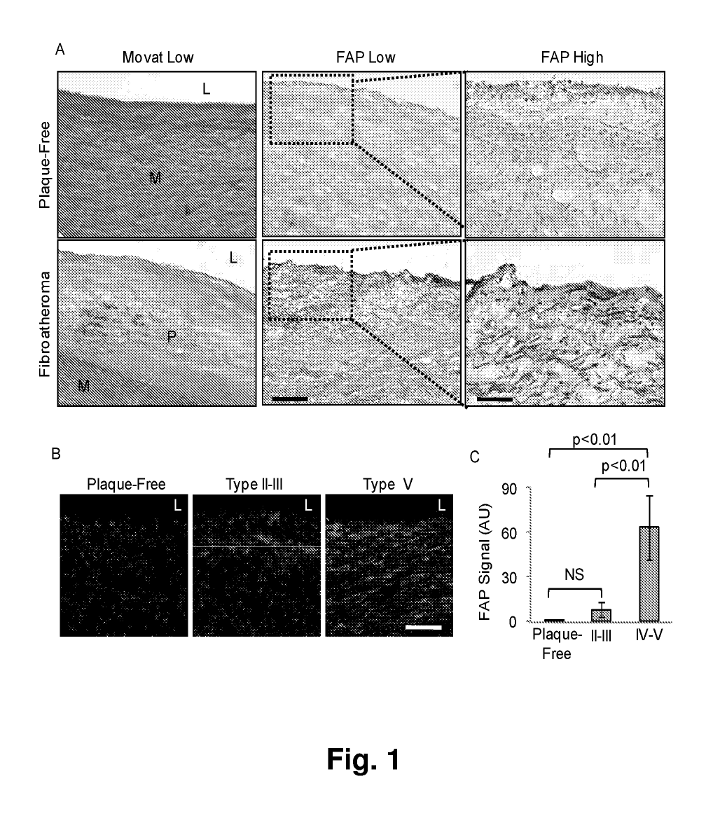

Immunofluorescent stainings for FAP in adjacent cryosections reveal enhanced expression in fibroatheromata vs. plaque-free aortae (FIG. 1A), as characterized by the AHA grading criteria. Positive staining for FAP is virtually absent in healthy ascending aortae whereas a step-wise increase is observed in Type II-III and Type IV-V plaques (FIG. 1B). Quantitative image analysis showed that FAP is significantly enhanced in advanced aortic plaques as compared to plaque-free aortae or early plaques (FIG. 1C).

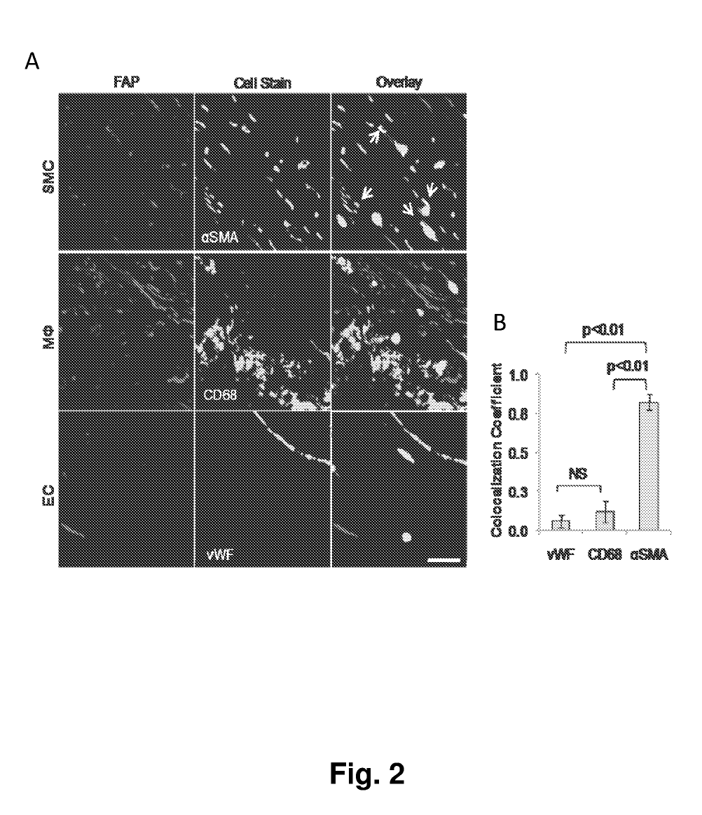

To characterize FAP-expressing cell types in human atherosclerotic plaques, immunofluorescent co-staining of FAP in macrophages is performed (identified as CD68-positive cells), smooth muscle cells (αSMA-positive cells), and endothelial cells (vWF-positive cells) (FIG. 2A). Confocal image analyses reveals that FAP expression by smooth muscle cells, but not by macrophages or endothelial cells (FIG. 2B).

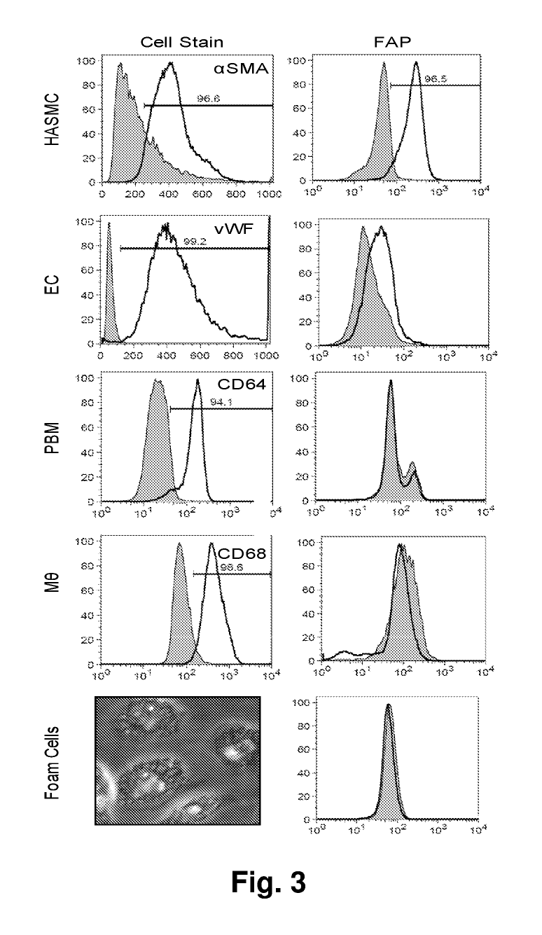

To validate FAP expression by vascular cells in vitro, FACS analyses of FAP in HASMC (αSMA-positive cells), HAEC (vWF-positive cells), peripheral blood derived-monocytes (CD64-positive), macrophages (CD68-positive), and foam cells (Oil-Red-O-positive macrophages) is performed. FACS analyses reveals high constitutive FAP expression in HASMC, slight expression in HAEC, but no expression by peripheral blood-derived monocytes, macrophages, or foam cells (FIG. 3).

### Example 2

### FAP Expression is Enhanced in “Unstable” Thin Versus “Stable” Thick Fibrous Caps of Human Coronary Atheromata

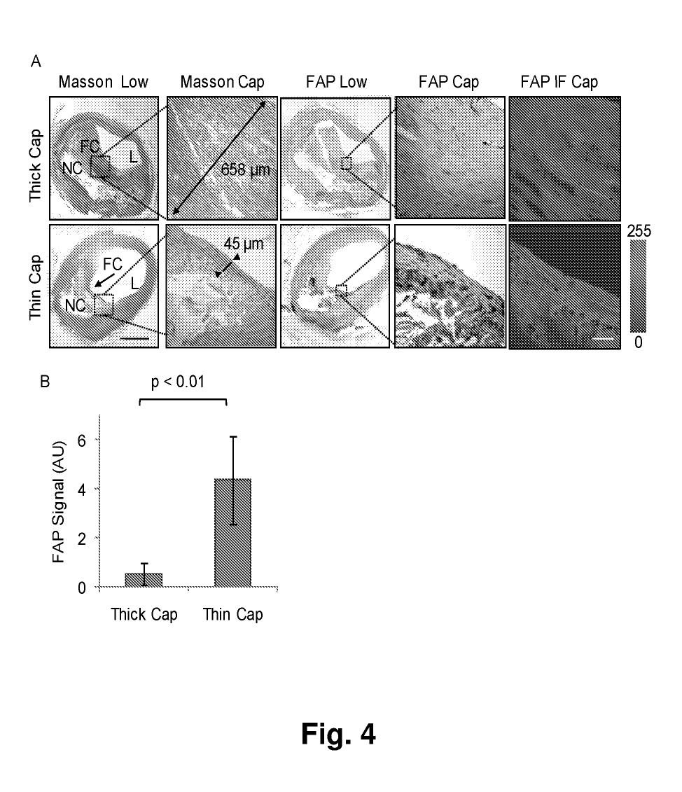

In order to determine the association of FAP with the degree of plaque vulnerability in clinically relevant atherosclerotic lesions, Masson method (stains collagen in blue) is performed of rupture-prone human coronary arteries obtained from patients that died after an acute myocardial infarction. Based on fibrous cap thickness, these specimens were characterized as “unstable” thin cap (<50 μm) or “stable” thick cap (>65 μm) fibroatheromata. Immunohistological and immunofluorescent stainings in adjacent sections revealed enhanced FAP expression in “unstable” thin vs. “stable” thick fibrous caps (FIG. 4A). Confocal image analyzes show a significant increase of FAP expression in “unstable” thin vs. “stable” thick fibrous caps of human coronary fibroatheromata (FIG. 4B).

### Example 3

### FAP Expression Associates with Macrophage Burden in Human Aortic Atherosclerotic Plaques

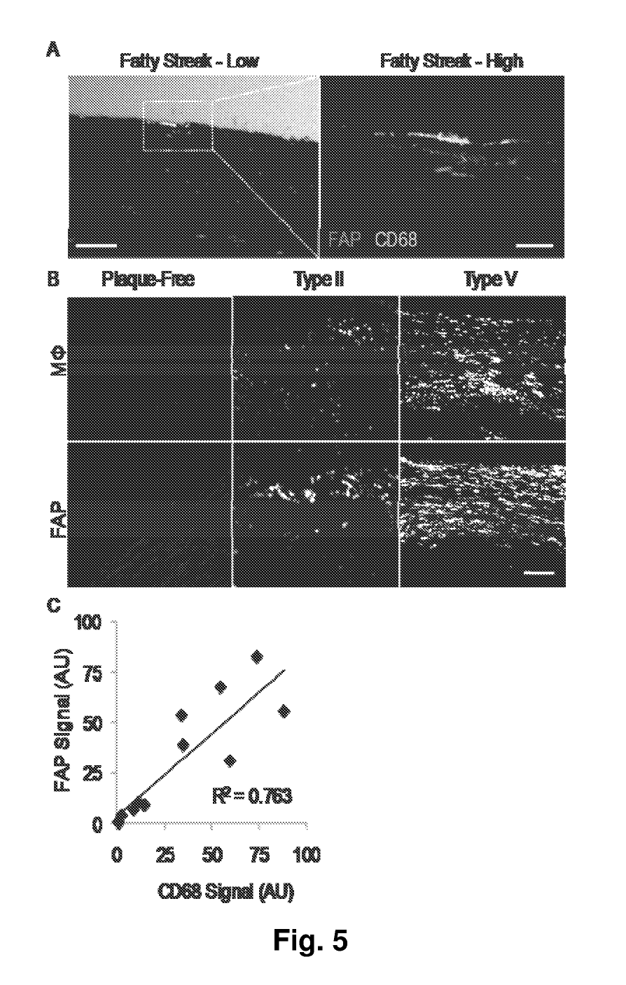

Immunofluorescence staining reveals FAP expression in medial cells adjacent to macrophages in a representative aortic fatty streak (FIG. 5A). For characterizing the relationship between FAP and inflammation, FAP and macrophage immunofluorescent signal intensity in human aortic plaques (FIG. 5B) is compared. A positive correlation between macrophage burden and FAP expression at multiple stages of plaque progression is found (R²=0.763; n=12; FIG. 5C).

### Example 4

### Macrophage-Derived TNFα Induces FAP Expression in Cultured Human Aortic Smooth Muscle Cells

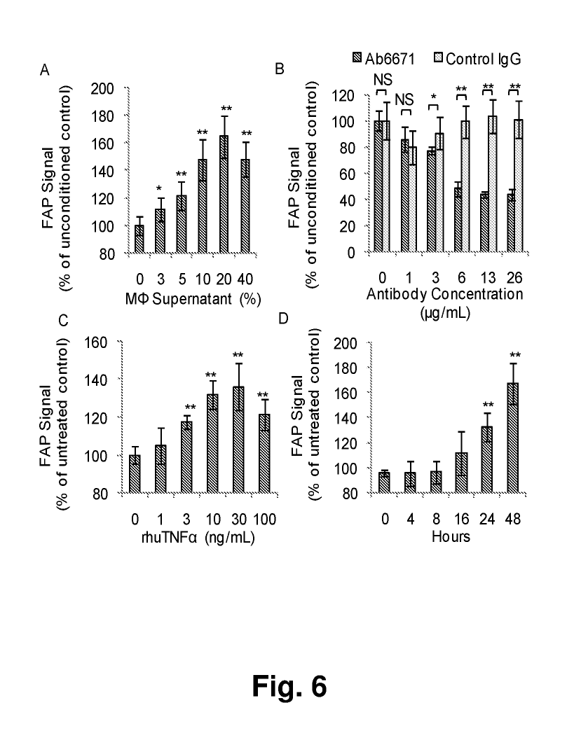

To elucidate a signaling mechanism between macrophages and FAP expressing HASMC, HASMC to macrophage-conditioned media for 48 hr is exposed to simulate conditions applicable to plaque inflammation. Cultured HASMC show a significant dose-dependent increase in FAP in response to the macrophage-conditioned media with a maximal effect observed at 20% media concentration (FIG. 6A) after 48 hr. This effect is abolished when macrophage-conditioned media is treated with a TNFa-neutralizing antibody (FIG. 6B). To confirm paracrine-induced FAP expression by TNFα, recombinant human TNFα is used to induce a FAP in HASMC in a dose- and time-dependent manner, with a maximum response at 30 ng/mL for 48 hr (FIGS. 6C and 6D).

### Example 5

### Gelatinolytic Activity in Aortic Fibrous Caps is Inhibited by an FAP Neutralizing Antibody

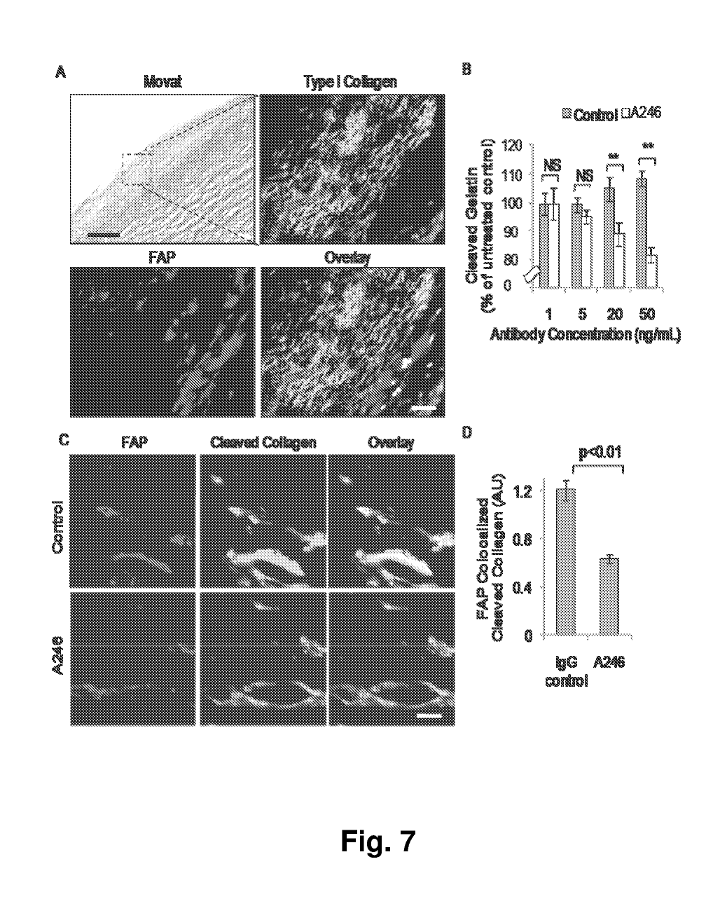

Immunofluorescence analyses revealed enhanced FAP expression in Type I collagen-poor regions of aortic fibrous caps (FIG. 7A). Recombinant human FAP degrades gelatin in a dose- and time-dependent manner (FIG. 7B). A246, an antibody directed against FAP's catalytic site, reduce the gelatinolytic activity of both recombinant human FAP and activated HASMC (FIGS. 7C & 7D). Aortic fibrous caps treated with an IgG control antibody show a colocalization of FAP with cleaved collagen type I, whereas Ab246-treated plaques demonstrate a significantly reduced colocalization of FAP with type I collagenase activity (FIG. 7E). Confocal image analysis reveal that A246-treated fibrous caps exhibit decreased cleaved type I collagen at sites of FAP expression (FIG. 7F).

### Example 6

### FAP Expression is in Enhanced in Aortic Atherosclerotic Plaques of ApoE^{-/-} Mice

Plaques are analyzed in aortic roots of male atherosclerotic apolipoprotein E knockout (ApoE^{-/-}) mice (C57BL/6J background) fed a high-cholesterol diet (1.25% total cholesterol; RD12108 from Research Diets) for 12 weeks, starting at 8 weeks of age (n=5). Wild-type (WT) male C57BL/6J mice on a normal diet for the same period are used as healthy controls (n=4). The aortic roots of mice euthanized by isoflurane are rinsed with normal saline, excised and immediately embedded in OCT, and frozen for sectioning. The confirmed presence of FAP in atherosclerotic plaques of ApoE^{-/-} knockout mice validates the use of this model for in-vivo FAP inhibition studies by intravenous injection of FAP neutralizing antibodies (FIG. 12).

### Example 7

### A Sandwich ELISA has been Established to Quantify Soluble FAP in Human Peripheral Blood

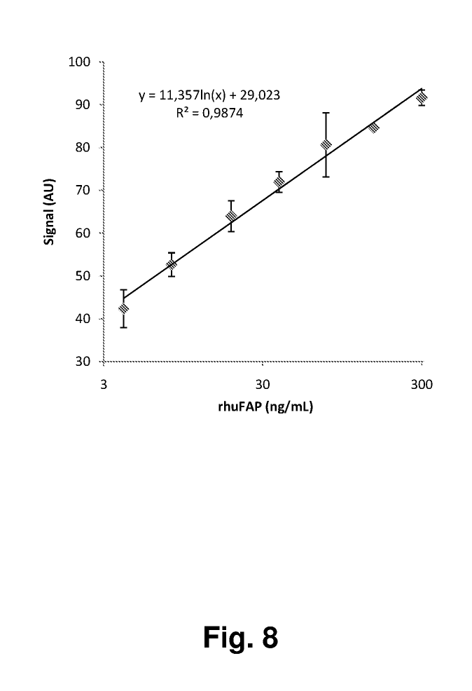

Sandwich ELISA can be performed using standard protocols using U-shaped transparent 96-well microtiter plates, wherein anti-FAP antibodies can be coated with the plate in order to detect FAP protein. 100 μl of recombinant FAP protein (rhuFAP) solution at a concentration of 3 to 300 ng/ml is added to the wells and are incubated for 1 h at 25° C. After incubation of the samples the wells are washed twice, 100 μL per well of the first detection antibody (mAb FAP) can be added and incubated in the plate (1 h, 25° C.). After 3 washing steps 100 μL of the secondary fluorescence detection antibody (diluted 1:3000) can be added and incubated (1 h at 25° C.). After the incubation, the wells are washed 3 times. All samples run in triplicate and the results are compared against the standard curve. The assay sensitivity level and the range were determined using 3-300 ng of purified human FAP (FIG. 8). To simulate the natural background, 1 μg of E. coli whole cell extract can be added to each sample. This number corresponds to signal which is above the average of the background signal for three times of the blank signal standard deviation. The dependence observed between the FAP amount and the fluorescent signal can be used for FAP concentration estimation in real samples. The results are shown in FIG. 8.

### Example 8

### FAP Accelerates the Rate of Human Blood Clotting

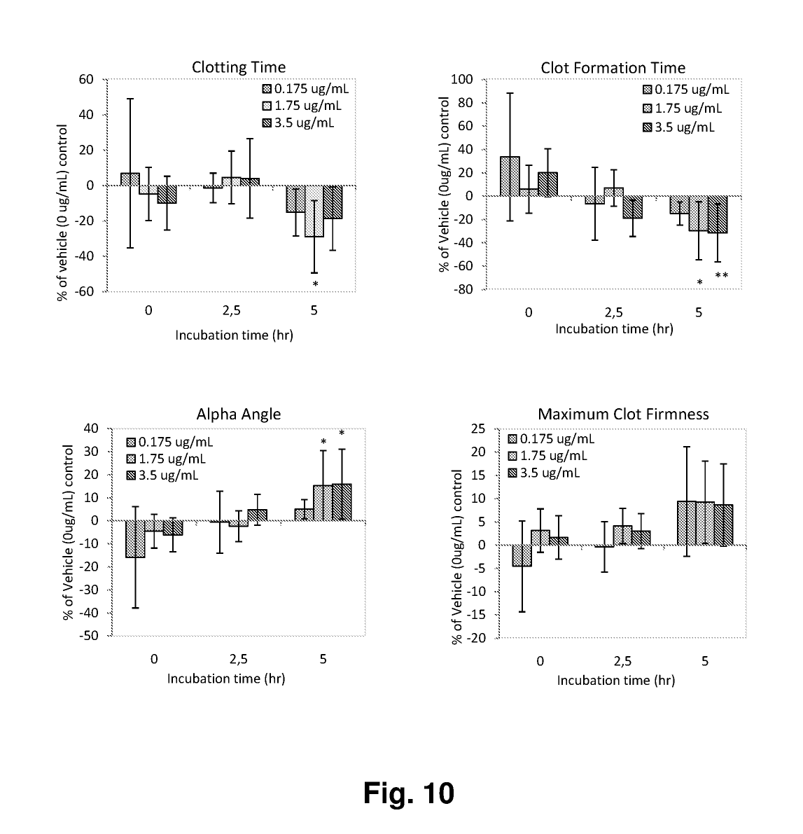

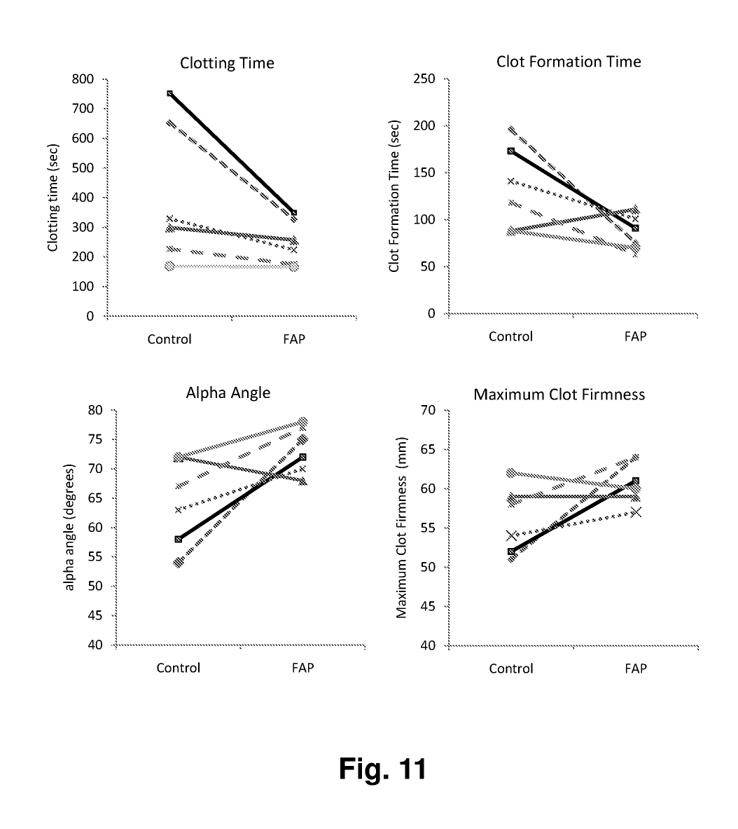

The ROTEM/NATEM (ROTEM®; Pentapharm CO, Munich, Germany) technique is used according to the manufactures instructions. Peripheral blood samples are harvested in citrate tubes from healthy probands and conditioned with recombinant human FAP (rhu FAP 0, 0.875 μg/ml, 1.750 μg/ml and 3.5 μg/ml) for 0 up to 5 h. Control blood samples are incubated without recombinant FAP. Representative ROTEM readouts from a NATEM assay revealed that FAP accelerates the rate of human blood clotting (FIG. 9). In addition, clotting time, clot formation time, alpha angle and clot firmness are increased in healthy patients blood samples treated with recombinant human FAP. Blood from 6 healthy probands exhibiting an endogenous plasma FAP level below 20 ng/mL are harvest and conditioned with recombinant human FAP at a doses of 0.175 μg/ml, 1.75 μg/ml and 3.5 μg/ml and incubated for 0, 2.5 h and 5 h. Thereby it is observed that FAP accelerates clotting time and clot formation time and enhances alpha angle and maximum clot firmness (FIG. 10). These results are further confirmed by applying the NATEM technique. Samples are treated as above described. Thus revealing that absolute clotting time and clot formation time decreased in 5/6 probands, alpha angle increased in 5/6 patients, and maximum clot thickness increased in 4/6 probands when conditioned with 1.75 ug/mL of FAP at 5 hr incubation compared to vehicle treated control (FIG. 11).

### Example 9

### FAP is Expressed in the Endothelium of Vulnerable Thin-Cap Coronary Fibroatheromata

Cross sections from human paraffin-embedded coronary plaque and internal mammary arteries (4 μm thickness) were mounted on glass slides. Antigen retrieval in paraffin-embedded sections was performed after 20 min incubation in 95° C. retrieval buffer (2 mM Sodium Citrate, pH 7.6). FAP was stained using a rabbit polyclonal antibody raised against the catalytic insert of FAP (A246; Ab28246; Abcam, Cambridge, Mass.) and a rabbit isotype control (Ab37415; Abcam). CD31 was stained using a mouse monoclonal antibody (303108; Biolegend) and matching isotype control (400123; Biolegend). Primary antibodies were detected with biotin-labeled goat anti-mouse (115-066-003; Jackson ImmunoResearch, West Grove, Pa.) and biotin-labeled goat anti-rabbit (111-066-003; Jackson ImmunoResearch) and stained using an ABC staining kit (Vector Labs, Burlingame, Calif.).

In order to determine FAP expression in the endothelium of human coronary fibrous caps, we stained H&E stainings in rupture-prone human coronary arteries obtained from patients that died after myocardial infarction and healthy internal mammary arteries as control. Immunohistological stainings revealed FAP expression in the fibroatheromata endothelium (FIG. 13).

### Example 10

### FAP Expression is Enhanced in Human Coronary Thrombi Compared to Peripheral Blood

Cross sections from human paraffin-embedded thrombi and peripheral blood sections (4 μm thickness) were mounted on glass slides. Antigen retrieval in paraffin-embedded sections of coronary thrombi and peripheral blood was performed after 20 min incubation in 95° C. retrieval buffer (2 mM Sodium Citrate, pH 7.6). FAP was stained using a rabbit polyclonal antibody raised against the catalytic insert of FAP (A246; Ab28246; Abcam, Cambridge, Mass.) and a rabbit isotype control antibody (Ab37415; Abcam). Primary antibodies were detected with a biotin-labeled goat anti-rabbit (111-066-003; Jackson ImmunoResearch) antibody, and stained using an ABC staining kit (Vector Labs, Burlingame, Calif.). Immunohistochemical stainings for FAP in adjacent paraffin-embedded sections reveal enhanced FAP expression in human coronary thrombi vs. peripheral blood (FIG. 14).

### Example 11

### FAP is Expressed by Granulocytes in Human Coronary Thrombi

Thrombi from the coronary artery were aspirated from patients suffering from myocardial infarction. 5 mL of peripheral blood was drawn from each patient into the citrated tube less than one minute prior to thrombus aspiration. Peripheral blood (1 mL) and coronary thrombus material were placed into 1 mL of Acutase with 50 μL Actilyse, and shaken gently at 37° C. for 1 hr. Cell aggregates were further dissociated by sifting through a cell strainer (40 μm pore size) using the soft rubber from a syringe. Both samples were then spun at 400 G for 5 min and the supernatant removed. Cell pellets were then resuspended in FACS buffer (PBS with 1% FCS and 5 mM EDTA) with 1 μg/mL for Fc receptor blocking agent and incubated for 30 min at 4° C. Cells were subsequently labeled with fluorescently tagged antibodies against FAP (clone F19), Granulocytes (CD66b; Biolegend 555724), Monocytes (CD45; Biolegend 345809), and T-Lymphocytes (CD3; Biolegend 555332).

Flow cytometry is used to quantify FAP expression in human peripheral blood vs. thrombus leukocyte cell populations including; monocytes, T-Cells, and granulocytes. Quantitative analyses revealed enhanced FAP expression in thrombus granulocytes, but not in the other analyzed cell populations (FIG. 15A-F).

### Example 12

### FAP Expression in Granulocytes is Enhanced in Thrombi of STEMI, but Not PAOD Patients

Thrombi from the femoral artery were aspirated from patients suffering from peripheral artery occlusive disease. 5 mL of peripheral blood was drawn from each patient into the citrated tube seconds prior to thrombus aspiration. The thrombi and blood specimens were placed in phosphate buffered saline and transferred to the laboratory for processing. POAD thrombus specimens were prepared in accordance to the description provided in Example 11 above.

To access whether granulocyte-specific FAP expression associates with acute atherothrombosis, we used flow cytometry to quantify FAP expression in peripheral blood and occluding thrombi from patients suffering from ST segment elevation myocardial infarction (STEMI) (coronary artery thrombi) and peripheral artery occlusive disease (PAOD) (femoral artery thrombi). Acutely formed coronary thrombi showed increased expression of FAP compared to chronic peripheral artery derived thrombi (FIG. 16).

### Example 13

### Plasma FAP Levels are Enhanced in Patients with Acute Coronary Syndromes (ACS)

5 mL of blood was drawn into a citrate tube from patients with no coronary artery disease (CAD), stable angina pectoris, unstable angina pectoris, and STEMI. Blood was spun for 10 min at 400G and the plasma layer was extracted and frozen at −80 C for later analyses. A Sandwich ELISA for soluble FAP was performed as follows. A rabbit polyclonal antibody raised against the catalytic insert of FAP (A246; Ab28246; Abcam, Cambridge, Mass.) was used as a capture antibody, and mAb FAP was used as the detection antibody. Human recombinant FAP protein (rhuFAP) solution at a concentration of 3 to 5000 ng/ml is added to the wells to establish the standard curve. After coating with the capture antibody, the plate was incubated with the plasma samples and the standards for 1 h at 25° C. After incubation of the samples the wells were washed twice with 100 μL of PBS per well. The detection antibody (mAb FAP) was be added and incubated in the plate (1 h, 25° C.). After 3 washing steps 100 μL of the secondary HRP-labeled detection antibody was added and incubated (1 h at 25° C.). After the incubation, the wells were washed 3 times and Peroxidase Substrate was added to generate the signal for detection. The resulting signal was read using a plate reader to determine the FAP concentration in the plasma samples. ELISA analysis reveals enhanced plasma levels of FAP in patients with ACS compared to patients with no CAD (FIG. 17).

### CONCLUSION

The fibrous cap of an atherosclerotic plaque is essential for separating the lumen from the thrombogenic necrotic core. Since the mechanical strength of the fibrous cap is provided by collagen, MMPs and cysteine proteases that degrade collagen are associated with plaque instability and occurrence of acute thrombotic events.²⁹⁻³³ This study links the constitutively active serine protease FAP to plaque progression and vulnerability and shows that: (1) FAP expression is enhanced in human aortic atheromata and in fibrous caps of rupture-prone coronary arteries, (2) FAP is expressed in HASMC and correlates with inflammatory macrophage burden; (3) FAP is induced in HASMC by macrophage-derived TNFα via paracrine signaling, (4) FAP cleaves collagen in fibrous caps of human atheromata, and (5) FAP-mediated collagenase activity is inhibited by an FAP-blocking antibody in human fibrous caps.

Numerous studies implicate matrix-degrading collagenases such as MMP1, 2, and 9 as well as cysteine proteases such as cathepsins S and K in vascular remodeling and plaque rupture.^{13,30,34} The findings presented here provide evidence that FAP is the first known smooth muscle cell-derived serine protease involved in collagen degradation in human atherosclerosis. FAP expression is particularly enhanced in fibrous caps of rupture-prone human coronary arteries isolated from patients dying after an acute myocardial infarction. FAP's deleterious collagenolytic activity is evidenced by FAP-mediated collagenolysis in fibrous caps and its colocalization in collagen poor fibrous cap tissue. These findings enhance the potential clinical relevance of these findings that may be extended to patients rupture prone plaque and acute coronary syndromes (ACS).

Inflammation is another key feature of plaque vulnerability that has been shown to induce collagenases in atherosclerotic plaques.^{4,35,36} Consistent with this paradigm, we show that FAP expression in HASMC associates with macrophage burden in intermediate and advanced human atherosclerotic plaques. FAP is not expressed by macrophages. However, macrophage-derived TNFα induced a dose- and time-dependent increase in FAP expression in cultured smooth muscle cells. These results indicate that FAP is induced in smooth muscle cells by a paracrine-mediated inflammatory pathway, and thereby contributes to a growing body of evidence which supports the notion of inflammation-induced collagenase expression in atherosclerosis.^{6,11,29,37} Such findings could motivate future studies to investigate atheroprotective interventions against key anti-inflammatory mechanisms such as the TNFα pathway.

In addition to its expression in smooth muscle cells, constitutive FAP expression can also be detected in human aortic endothelial cells in vitro. Endothelial activation is a critical step in atherogenesis.³⁸ Activated endothelial cells express fibrous cap-degrading collagenases per se, and have also been shown to act in concert with fibrous cap degrading smooth muscle cells.5,32,39 The observed capacity of endothelial cells to express FAP supports this notion of a coordinated remodeling of the fibrous cap by both endothelial and smooth muscle cells. Furthermore, by promoting recruitment of blood-borne inflammatory cells to the plaque, activated endothelial cells may also enhance macrophage-derived cytokine release, activate smooth muscle cells, and thus induce FAP. Therefore, activated smooth muscle and endothelial cells may act as “partners in crime” in promoting plaque instability.

Atherogenic proteases exhibit enzymatic behavior which may be exploited for in vivo molecular imaging of vulnerable atherosclerotic plaques.⁴⁰ Indeed, recent optical imaging studies have utilized protease-specific fluorochrome-labeled peptides, which emit amplified signals after cathepsin K enzymatic cleavage, thereby increasing locally the signal-to-noise ratio for in vivo imaging.^{8,9,41} As a protease associated with human coronary plaque vulnerability, FAP might share this potential as a target for future in vivo molecular imaging studies. While FAP-mediated gelatinase activity can be detected in vitro, future studies are needed to determine the feasibility of FAP-based molecular imaging.

Expression of FAP in rupture-prone coronary plaques and endothelial cells motivates to extend further investigations to patients with ACS. The present study demonstrates that FAP-mediated lysis is induced by inflammatory signaling and can be neutralized by a blocking antibodies, thereby motivating future FAP-targeting feasibility studies in atherosclerosis and other FAP-related inflammatory diseases such as rheumatoid arthritis and tumor formation.

### REFERENCES

1. Farb A, Burke A P, Tang A L, Liang Y, Mannan P, Smialek J, Virmani R. Coronary plaque erosion without rupture into a lipid core: A frequent cause of coronary thrombosis in sudden coronary death. Circulation. 1996; 93:1354-1363
2. Virmani R, Burke A P, Farb A, Kolodgie F D. Pathology of the vulnerable plaque. Journal of the American College of Cardiology. 2006; 47:C13-C18
3. van der Wal A, Becker A, van der Loos C, Das P. Site of intimal rupture or erosion of thrombosed coronary atherosclerotic plaques is characterized by an inflammatory process irrespective of the dominant plaque morphology. Circulation. 1994; 89:36-44
4. Shah P, Falk E, Badimon J, Fernandez-Ortiz A, Mailhac A, Villareal-Levy G, Fallon J, Regnstrom J, Fuster V. Human monocyte-derived macrophages induce collagen breakdown in fibrous caps of atherosclerotic plaques. Potential role of matrix-degrading metalloproteinases and implications for plaque rupture. Circulation. 1995; 92:1565-1569
5. Rajavashisth T B, Xu X-P, Jovinge S, Meisel S, Xu X-O, Chai N-N, Fishbein M C, Kaul S, Cercek B, Sharifi B, Shah P K. Membrane type 1 matrix metalloproteinase expression in human atherosclerotic plaques. Circulation. 1999; 99:3103-3109
6. Crisby M, Nordin-Fredriksson G, Shah P K, Yano J, Zhu J, Nilsson J. Pravastatin treatment increases collagen content and decreases lipid content, inflammation, metalloproteinases, and cell death in human carotid plaques: Implications for plaque stabilization. Circulation. 2001; 103:926-933
7. Herman M P, Sukhova G K, Libby P, Gerdes N, Tang N, Horton D B, Kilbride M, Breitbart R E, Chun M, Schonbeck U. Expression of neutrophil collagenase (matrix metalloproteinase-8) in human atheroma: A novel collagenolytic pathway suggested by transcriptional profiling. Circulation. 2001; 104:1899-1904
8. Aikawa E, Nahrendorf M, Figueiredo J-L, Swirski F K, Shtatland T, Kohler R H, Jaffer F A, Aikawa M, Weissleder R. Osteogenesis associates with inflammation in early-stage atherosclerosis evaluated by molecular imaging in vivo. Circulation. 2007; 116:2841-2850
9. Jaffer F A, Kim D-E, Quinti L, Tung C-H, Aikawa E, Pande A N, Kohler R H, Shi G-P, Libby P, Weissleder R. Optical visualization of cathepsin k activity in atherosclerosis with a novel, protease-activatable fluorescence sensor. Circulation. 2007; 115:2292-2298
10. Schafers M, Riemann B, Kopka K, Breyholz H-J, Wagner S, Schafers K P, Law M P, Schober O, Levkau B. Scintigraphic imaging of matrix metalloproteinase activity in the arterial wall in vivo. Circulation. 2004; 109:2554-2559
11. Deguchi J-o, Aikawa M, Tung C-H, Aikawa E, Kim D-E, Ntziachristos V, Weissleder R, Libby P. Inflammation in atherosclerosis: Visualizing matrix metalloproteinase action in macrophages in vivo. Circulation. 2006; 114:55-62
12. Herman M P, Sukhova G K, Kisiel W, Foster D, Kehry M R, Libby P, Schönbeck U. Tissue factor pathway inhibitor-2 is a novel inhibitor of matrix metalloproteinases with implications for atherosclerosis. The Journal of Clinical Investigation. 2001; 107: 1117-1126
13. Sukhova G K, Shi G P, Simon D I, Chapman H A, Libby P. Expression of the elastolytic cathepsins s and k in human atheroma and regulation of their production in smooth muscle cells. The Journal of Clinical Investigation. 1998; 102:576-583
14. Kong Y-Z, Yu X, Tang J-J, Ouyang X, Huang X-R, Fingerle-Rowson G, Bacher M, Scher L A, Bucala R, Lan H Y. Macrophage migration inhibitory factor induces mmp-9 expression: Implications for destabilization of human atherosclerotic plaques. Atherosclerosis. 2005; 178:207-215
15. Bauer S, Jendro M, Wadle A, Kleber S, Stenner F, Dinser R, Reich A, Faccin E, Godde S, Dinges H, Muller-Ladner U, Renner C. Fibroblast activation protein is expressed by rheumatoid myofibroblast-like synoviocytes. Arthritis Research & Therapy. 2006; 8:R171
16. Edosada C Y, Quan C, Wiesmann C, Tran T, Sutherlin D, Reynolds M, Elliott J M, Raab H, Fairbrother W, Wolf B B. Selective inhibition of fibroblast activation protein protease based on dipeptide substrate specificity. J. Biol. Chem. 2006; 281:7437-7444
17. Park J E, Lenter M C, Zimmermann R N, Garin-Chesa P, Old U, Rettig W J. Fibroblast activation protein, a dual specificity serine protease expressed in reactive human tumor stromal fibroblasts. Journal of Biological Chemistry. 1999; 274:36505-36512
18. Levy M T, McCaughan G W, Abbott C A, Park J E, Cunningham A M, Miller E, Rettig W J, Gorrell M D. Fibroblast activation protein: A cell surface dipeptidyl peptidase and gelatinase expressed by stellate cells at the tissue remodelling interface in human cirrhosis. Hepatology. 1999; 29:1768-1778
19. Aertgeerts K, Levin I, Shi L, Snell G P, Jennings A, Prasad G S, Zhang Y, Kraus M L, Salakian S, Sridhar V, Wijnands R, Tennant M G. Structural and kinetic analysis of the substrate specificity of human fibroblast activation protein α. Journal of Biological Chemistry. 2005; 280:19441-19444
20. Stary H C, Chandler A B, Dinsmore R E, Fuster V, Glagov S, Insull W, Jr, Rosenfeld M E, Schwartz C J, Wagner W D, Wissler R W. A definition of advanced types of atherosclerotic lesions and a histological classification of atherosclerosis: A report from the committee on vascular lesions of the council on arteriosclerosis, american heart association. Circulation. 1995; 92:1355-1374
21. Stary H C, Chandler A B, Dinsmore R E, Fuster V, Glagov S, Insull W, Jr, Rosenfeld M E, Schwartz C J, Wagner W D, Wissler R W. A definition of advanced types of atherosclerotic lesions and a histological classification of atherosclerosis: A report from the committee on vascular lesions of the council on arteriosclerosis, american heart association. Arterioscler Thromb Vasc Biol. 1995; 15:1512-1531
22. Rettig W J, Pilar G-C, Beresford H R, Oettgen H F, Melamed M R, Old U. Cell-surface glycoproteins of human sarcomas: Differential expression in normal and malignant tissues and cultured cells. Proc. Natl. Acad. Sci. USA. 1988; 85:3110-3114
23. Dippold W G, Lloyd K O, Li Lucy T C, Ikeda H, Oettgen H F. Cell surface antigens of human malignant melanoma: Definition of six antigenic systems with mouse monoclonal antibodies. Proc. Natl. Acad. Sci. USA. 1980; 77:6114-6118
24. Smith P D, McLean K J, Murphy M A, Wilson Y, Murphy M, Turnley A M, Cook M J. A brightness-area-product-based protocol for the quantitative assessment of antigen abundance in fluorescent immunohistochemistry. Brain Research Protocols. 2005; 15:21-29
25. Landmann L, Marbet P. Colocalization analysis yields superior results after image restoration. Microscopy Research and Technique. 2004; 64:103-112
26. Mosedale D, Metcalfe J, Grainger D. Optimization of immunofluorescence methods by quantitative image analysis. J. Histochem. Cytochem. 1996; 44:1043-1050
27. Zinchuk V, Zinchuk O, Okada ^{T}. Quantitative colocalization analysis of multicolor confocal immunofluorescence microscopy images: Pushing pixels to explore biological phenomena. Acta Histochem Cytochem. 2007; 40:101-111
28. Agnati L F, Fuxe K, Torvinen M, Genedani S, Franco R, Watson S, Nussdorfer G G, Leo G, Guidolin D. New methods to evaluate colocalization of fluorophores in immunocytochemical preparations as exemplified by a study on a2a and d2 receptors in chinese hamster ovary cells. J. Histochem. Cytochem. 2005; 53:941-953
29. Hansson G K. Inflammation, atherosclerosis, and coronary artery disease. N Engl J. Med. 2005; 352:1685-1695
30. Galis Z S, Khatri J J. Matrix metalloproteinases in vascular remodeling and atherogenesis: The good, the bad, and the ugly. Circ Res. 2002; 90:251-262
31. Newby A C. Dual role of matrix metalloproteinases (matrixins) in intimal thickening and atherosclerotic plaque rupture. Physiol. Rev. 2005; 85:1-31
32. Sluijter J P G, Pulskens W P C, Schoneveld A H, Velema E, Strijder C F, Moll F, de Vries J-P, Verheijen J, Hanemaaijer R, de Kleijn D P V, Pasterkamp G. Matrix metalloproteinase 2 is associated with stable and matrix metalloproteinases 8 and 9 with vulnerable carotid atherosclerotic lesions: A study in human endarterectomy specimen pointing to a role for different extracellular matrix metalloproteinase inducer glycosylation forms. Stroke. 2006; 37:235-239
33. Sukhova G K, Schonbeck U, Rabkin E, Schoen F J, Poole A R, Billinghurst R C, Libby P. Evidence for increased collagenolysis by interstitial collagenases-1 and -3 in vulnerable human atheromatous plaques. Circulation. 1999; 99:2503-2509
34. Lutgens S P M, Cleutjens K B J M, Daemen M J A P, Heeneman S. Cathepsin cysteine proteases in cardiovascular disease. FASEB J. 2007; 21:3029-3041
35. Brown D L, Hibbs M S, Kearney M, Loushin C, Isner J M. Identification of 92-kd gelatinase in human coronary atherosclerotic lesions: Association of active enzyme synthesis with unstable angina. Circulation. 1995; 91:2125-2131
36. Aikawa M, Rabkin E, Sugiyama S, Voglic S J, Fukumoto Y, Furukawa Y, Shiomi M, Schoen F J, Libby P. An hmg-coa reductase inhibitor, cerivastatin, suppresses growth of macrophages expressing matrix metalloproteinases and tissue factor in vivo and in vitro. Circulation. 2001; 103:276-283
37. Spagnoli L G, Bonanno E, Mauriello A, Palmieri G, Partenzi A, Sangiorgi G, Crea F. Multicentric inflammation in epicardial coronary arteries of patients dying of acute myocardial infarction. J Am Coll Cardiol. 2002; 40:1579-1588
38. Szmitko P E, Wang C-H, Weisel R D, de Almeida J R, Anderson T J, Verma S, New markers of inflammation and endothelial cell activation: Part i. Circulation. 2003; 108:1917-1923
39. Skinner M P, Raines E W, Ross R. Dynamic expression of alpha 1 beta 1 and alpha 2 beta 1 integrin receptors by human vascular smooth muscle cells. Alpha 2 beta 1 integrin is required for chemotaxis across type i collagen-coated membranes. The American Journal of Pathology. 1994:1070-1081
40. Matter C M, Stuber M, Nahrendorf M. Imaging of the unstable plaque: How far have we got? Eur Heart J. 2009; 30:2566-2574
41. Aikawa E, Aikawa M, Libby P, Figueiredo J-L, Rusanescu G, Iwamoto Y, Fukuda D, Kohler R H, Shi G-P, Jaffer F A, Weissleder R. Arterial and aortic valve calcification abolished by elastolytic cathepsin s deficiency in chronic renal disease. Circulation. 2009; 119:1785-1794

1. A method of determining the presence of an inflammatory and/or cardiovascular disease or condition in a patient comprising assaying
(i) a sample of a body fluid taken from said patient for expression of Fibroblast Activation Protein (FAP), wherein expression or an increased expression of said FAP compared to a control sample is indicative for the disease or condition in said patient; and/or
(ii) a sample of a thrombus or plaque taken from said patient for expression of FAP, wherein an increased expression of FAP as compared to its expression in a sample of a body fluid taken from said patient is indicative for the disease or condition.

2. The method of claim 1, wherein said body fluid is blood.

3. The method of claim 1, wherein said disease is selected from the group atherosclerosis, rheumatoid arthritis, stroke or an acute coronary syndrome such as myocardial infarction, heart attack, chronic liver disease, cerebral venous thrombosis, deep venous thrombosis or pulmonary embolism.

4. The method of claim 2, wherein the condition is vulnerable atherosclerotic plaques or atherothrombosis.

5. The method of claim 1, comprising assaying said sample for FAP protein or mRNA.

6. The method of claim 5, wherein said assay is an immunoassay.

7. The method of claim 6, wherein said assay is an immunohistochemical assay.

8. The method of claim 7, wherein said assay comprises fluorescence activated cytometry (FACS), indirect ELISA, sandwich ELISA or cell culture ELISA.

9. The method of claim 6, wherein said immunoassay comprises contacting said sample with a monoclonal or polyclonal antibody which binds specifically to FAP.

10. The method of claim 9, wherein said monoclonal antibody is a mouse anti-human antibody.

11. A method of treating a disease selected from the group atherosclerosis, rheumatoid arthritis, stroke or an acute coronary syndrome such as myocardial infarction, heart attack, chronic liver disease, cerebral venous thrombosis, deep venous thrombosis or pulmonary embolism, said method comprising administering to a patient a composition comprising a compound capable of inhibiting the activity of FAP or its expression.

12. The method of claim 11, wherein the composition is a pharmaceutical composition and comprises a pharmaceutically acceptable carrier.

13. The method of claim 11, wherein said activity is enzymatic activity.

14. The method of claim 13, wherein the composition inhibits blood clotting.

15. The method of claim 11, wherein said compound is capable of binding to FAP or its encoding nucleic acid molecule.

16. The method of claim 15, wherein said compound is a monoclonal antibody which specifically birds to FAP.

17. The method of claim 6, wherein said monoclonal antibody is a human antibody.

18. The method of claim 11, wherein said compound is a FAP antagonist.

19. The method of claim 18, wherein said FAP antagonist is a peptide or peptide analog.

20. The method of claim 11, wherein the patient has been diagnosed in accordance with a method comprising determining the presence of an inflammatory and/or cardiovascular disease or condition in the patient comprising assaying
(i) a sample of a body fluid taken from said patient for expression of Fibroblast Activation Protein (FAP), wherein expression or an increased expression of said FAP compared to a control sample is indicative for the disease or condition in said patient; and/or
(ii) a sample of a thrombus or plaque taken from said patient for expression of FAP, wherein an increased expression of FAP as compared to its expression in a sample of a body fluid taken from said patient is indicative for the disease or condition.

21. A method of preventing or treating a coagulation disorder, said method comprising administering to a patient a pharmaceutical composition comprising FAP.

27. The method of claim 21, wherein the coagulation disorder is hemophilia.

23. A method of using FAP as a reagent for enhancing blood clotting in vitro.

24. A method of using ROTEM/NATEM for testing the therapeutic effect and/or efficiency of FAP blocking agents in a patient.

25. A method of diagnosing a disease selected from the group atherosclerosis, rheumatoid arthritis, stroke or an acute coronary syndrome such as myocardial infarction, heart attack, chronic liver disease, cerebral venous thrombosis, deep venous thrombosis or pulmonary embolism using a composition comprising a compound capable of detecting the presence or activity of FAP.

26. The method of claim 25, wherein the composition is a diagnostic composition comprising a suitable means for detection of the compound and/or of FAP.
